(19) United States
(12) Patent Application Publication
(10) Pub. No.: US 2026/0000620 A1
(43) Pub. Date: Jan. 1, 2026

(54) ORAL DOSAGE FORMS

(71) Applicant: Amryt Endo, Inc., Boston, MA (US)

(72) Inventors: David CARON, Even Yehuda (IL); Isaac LANDAU, Efrat (IL)

(21) Appl. No.: 18/728,720

(22) PCT Filed: Jan. 13, 2023
(86) PCT No.: PCT/US2023/060631

§ 371 Date: Jul. 12, 2024

Related U.S. Application Data
(60) Provisional application No. 63/299,128, filed on Jan. 13, 2022.

(51) Int. Cl. A61K 9/48; A61K 38/22; A61K 38/31

(52) U.S. Cl. CPC A61K 9/4891; A61K 9/4816; A61K 9/4825; A61K 9/485; A61K 9/4858; A61K 9/4866; A61K 38/22; A61K 38/31

(57) ABSTRACT

The present invention relates generally to encapsulated pharmaceutical compositions enabling improved oral delivery and methods of using such compositions.

### CROSS-REFERENCE TO RELATED APPLICATIONS

This application is a U.S. National Stage Application filed under 35 U.S.C. § 371 of International Application No. PCT/US2023/060631, filed Jan. 13, 2023, which claims priority to U.S. Provisional Application No. 63/299,128, filed Jan. 13, 2022, the disclosures of which are hereby incorporated by reference in their entirety for all purposes.

### FIELD OF THE TECHNOLOGY

The present invention relates generally to encapsulated pharmaceutical compositions enabling improved oral delivery and methods of using such compositions

### BACKGROUND

The oral bioavailability of peptides and protein can be limited due to poor absorbance of the intact and active molecules from gastrointestinal tract to the blood. Therefore, to reach systemic exposure, peptide medications are usually injected. Chiasma's proprietary technology, the Transient Permeability Enhancer (TPE®), enables oral delivery of peptides.

A drug product is in the form of oily suspension, filled into enteric coated hard gelatin capsules. The active drug (octreotide), together with a permeation enhancer (sodium caprylate=NaC₈) and matrix forming excipients comprise the solid phase of the suspension. The drug product is called Mycapssa®—oral octreotide capsules for the treatment of acromegaly. Octreotide acetate is formulated in the Transient Permeability Enhancer (TPE®) excipient mixture to form an oily suspension of solid hydrophilic particles within a lipophilic medium. The oily suspension is filled into hard gelatin capsules which are then banded with gelatin and film-coated by an enteric coating system, enabling delivery of the capsule contents to the intestine. This has been described inter alia in co-assigned U.S. Pat. No. 8,329,198. It was proven that oral intake of Mycapssa delivers the required dose to the acromegaly patients and is sufficient to elicit GH and IGF-1 control in acromegaly patients.

It was desirable to produce a new product with improved pharmacokinetic properties and/or dosing regime, enabling higher doses of octreotide as needed for acromegaly and for other indications which require a higher dosage. This can also enable higher doses of other therapeutically active drugs.

It was therefore desirable to improve the level of the bioavailability of the polypeptides in the capsules.

### SUMMARY

The inventors of the present invention have discovered that the absorption of certain therapeutic agents such as polypeptides in a subject can be improved when administered in an oral dosage form with a coating as described herein.

One embodiment of the invention is an oral dosage form comprising a capsule comprising a therapeutic agent e.g. a polypeptide wherein the capsule is coated with a first coating comprising polyvinyl alcohol, and a second coating on top of the first coating comprising methacrylic acid and ethyl acrylate copolymer dispersion.

Another embodiment of the invention is an oral dosage form comprising a capsule comprising a therapeutic agent e.g. a polypeptide wherein the capsule is coated with a coating comprising methacrylic acid and ethyl acrylate copolymer dispersion.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments and are incorporated in and constitute a part of this specification. The drawings, together with the remainder of the specification, explain principles and operations of the described and claimed aspects and embodiments.

Throughout this application, various publications, including United States patents, are referenced by author and year and patents and applications by number. The disclosures of these publications and patents and patent applications in their entireties are hereby incorporated by reference into this application in order to more fully describe the state of the art to which this invention pertains.

### BRIEF DESCRIPTION OF THE DRAWINGS

Various aspects of at least one embodiment are discussed below with reference to the accompanying Figures. The Figures are provided for the purposes of illustration and explanation and are not intended as a definition of the limits of the invention.

In the Figures:

FIG. 1 presents Manufacturing flow chart for preparation of octreotide capsules as referenced in accompanying Example 1;

FIG. 2 presents Dissolution of prototypes at pH 6.8 as referenced in accompanying Example 2;

FIG. 3 presents Dog PK results of NC02 and NC04 against control (Mycapssa product) as referenced in accompanying Example 2;

FIG. 4 presents Manufacturing flow chart of capsules with new coating (NC04) as referenced in accompanying Example 3;

FIG. 5 presents Dissolution of batch OCT-CCP-035 at pH 6.8 as referenced in accompanying Example 3;

FIG. 6 presents Manufacturing flow chart of terlipressin capsules as referenced in accompanying Example 4; and

FIG. 7 presents Dissolution rate of terlipressin prototypes at pH 6.8 as referenced in accompanying Example 4.

### DETAILED DESCRIPTION OF THE INVENTION

The present invention relates generally to oral dosage forms comprising pharmaceutical compositions contained within an oral dosage form having a coating enabling improved delivery e.g. oral delivery and methods of using such compositions. Particular embodiments of the invention comprise an oral dosage form comprising the pharmaceutical composition, in particular an oral dosage form which is enteric coated with a coating enabling improved delivery. Further embodiments of the invention comprise a capsule containing the compositions of the invention, and in various embodiments the capsule is a hard gel or a soft gel capsule, and the capsule is enteric-coated as described herein. In other embodiments the coating is applied to pellets (microparticles or minitablets) which are then filled in a capsule such as a hard gel capsule or other dosage form or the pellets are compressed into tablets or even presented in a sachet form.

One embodiment of the invention is an oral dosage form comprising a capsule comprising a therapeutic agent e.g. a polypeptide wherein the capsule is coated with a first coating comprising polyvinyl alcohol, and a second coating on top of the first coating comprising methacrylic acid and ethyl acrylate copolymer dispersion (generally termed Eudragit® or Kollicoat®). A 3^{rd} coating (e.g., comprising talc) can be coated on top of the second coating.

Another embodiment of the invention is an oral dosage form comprising a capsule comprising a polypeptide wherein the capsule coating comprising methacrylic acid and ethyl acrylate copolymer dispersion (generally termed Eudragit or Kollicoat); talc can be an additional coating.

In another embodiment of the invention the ratio of methacrylic acid to ethyl acrylate in the dispersion is in the range 1.3:1 to 1:1.3 methacrylic acid to ethyl acrylate copolymer, or 1.2:1 to 1:1.2 methacrylic acid to ethyl acrylate copolymer or 1:1 methacrylic acid to ethyl acrylate copolymer.

In one embodiment of the invention the polyvinyl alcohol is partially hydrolysed and the polyvinyl alcohol has molecular weight between 20,000-35,000 preferably between 26,300 and 30,000.

In another embodiment of the invention the methacrylic acid and ethyl acrylate copolymer has molecular weight between 30,000 and 40,000 preferably about 34000 (average number molecular weight (Mn) is about 15000).

In one embodiment of the invention the polyvinyl alcohol is partially hydrolysed and the polyvinyl alcohol has an average molecular weight between 20,000-35,000 Da preferably between 26,300 and 30,000 Da.

In another embodiment of the invention the methacrylic acid and ethyl acrylate copolymer has an average molecular weight between 30,000 and 40,000 Da preferably about 34000 (average number molecular weight (Mn) is about 15000 Da).

In another embodiment there is a third coating on top of the second coating; this coating may comprise talc.

In another embodiment the capsule consists of gelatin or HPMC, in particular a hard gelatin capsule.

In another embodiment of the invention the second coating further comprises sodium lauryl sulfate and polysorbate 80 and does not comprise sodium bicarbonate or titanium dioxide.

In another embodiment of the invention the first coating which comprises polyvinyl alcohol (partially hydrolysed) further comprises talc, glycerol monocaprylocaprate type 1 and sodium lauryl sulfate; this is commercially available as OPADRY® ambII (Colorcon).

In another embodiment of the invention the second coating which comprises methacrylic acid and ethyl acrylate copolymer dispersion also comprises sodium lauryl sulfate and polysorbate 80; this is commercially available as Eudragit L30 D-55 (Evonik) or Kollicoat MAE 30DP (BASF). (Eudragit L30 D55 is the prepared suspension of Eudragit L100; Eudragit L100 is the polymer alone.)

In another embodiment of the invention the therapeutic agent is a polypeptide. In another embodiment of the invention the polypeptide is terlipressin or an analog thereof or octreotide or an analog thereof.

In another embodiment of the invention the polypeptide is terlipressin or salt thereof or octreotide or salt thereof usually octreotide acetate. Octreotide and octreotide acetate are used interchangeably herein.

In another embodiment of the invention the therapeutic agent e.g. a polypeptide e.g. octreotide or terlipressin is present in the oral dosage form in an amount of 1-50 mg, for example about 10-20 mg. In other embodiments of the invention the therapeutic agent e.g. a polypeptide is present in the oral dosage form in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 mg. In another embodiment of the invention the octreotide or terlipressin is present in the oral dosage form in an amount of 5-50 mg, preferably 10, 20 or 30 mg.

In another embodiment of the invention the therapeutic agent e.g. a polypeptide e.g. octreotide or terlipressin is present 1-50 mg per capsule, for example about 10-20 mg. In other embodiments of the invention the therapeutic agent e.g. a polypeptide is present at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 mg per capsule. In another embodiment of the invention the octreotide or terlipressin is present at 5-50 mg per capsule preferably 10, 20 or 30 mg.

Another embodiment of the invention is directed to a method of treatment of a subject suffering from hypotension, portal hypertension, variceal bleeding or hepatorenal syndrome or ascites which comprises administering to the subject a therapeutically effective amount of the oral dosage form of the invention comprising terlipressin as active pharmaceutical agent. The hypotension may be e.g., orthostatic hypotension or postprandial hypotension, the portal hypertension may comprise bleeding esophageal varices associated with portal hypertension and the ascites may be associated with liver cirrhosis and may be cirrhotic ascites or severe cirrhotic ascites; the hepatorenal syndrome (HRS) may be HRS I or HRS II. A subject may suffer from more than one of these conditions.

Another embodiment of the invention is directed to a method of treatment of a subject suffering from acromegaly or neuroendocrine tumor (NET), abnormal GI motility, carcinoid syndrome, flushing episodes associated with NET/carcinoid syndrome, portal hypertension, gastroparesis, diarrhea especially intractable diarrhea, diarrhea and/or flushing associated with NET/carcinoid syndrome, pancreatic leak or pancreatic pseudo-cysts, polycystic disease e.g. polycystic kidney disease or polycystic liver disease or PCOS or hypotension especially neurogenic orthostatic hypotension and postprandial hypotension which comprises administering to the subject a therapeutically effective amount of the oral dosage form of the invention

In another embodiment of the invention the oral dosage form of the invention comprising octreotide as active pharmaceutical agent is indicated for the long-term treatment of the severe diarrhea and flushing episodes associated with metastatic carcinoid tumors.

Another embodiment of the invention is a method of treatment of a subject which comprising administering any of the oral dosage forms described herein to the subject wherein the dosage is administered once, twice or three times per day; in another embodiment of the invention the administering occurs at least 1 hour before a meal or at least 2 hours after a meal; in another embodiment of the invention the administering occurs on an empty stomach. In one embodiment of the invention, one, two, three or four dosage forms may be administered simultaneously. In a particular embodiment, one or two dosage forms may be administered simultaneously.

Another embodiment of the invention is an oral dosage form where the first coating comprises 40-80% (wt %) of polyvinyl alcohol, 20-55% (wt %) of talc, 1-20% (wt %) of glycerol monocaprylate and 1-5% (wt %) of sodium lauryl sulfate and where the second coating comprises 80-99.0% (wt %) of methacrylic acid and ethyl acrylate copolymer, 0.1%-2% (wt %) of sodium lauryl sulfate and 0.5-4% (wt %) of polysorbate and additionally triethyl citrate.

Another embodiment of the invention is an oral dosage form where the first coating comprises 50-60% (wt %) of polyvinyl alcohol, 30-40% (wt %) of talc, 4-10% (wt %) of glycerol monocaprylate and 2-4% (wt %) of sodium lauryl sulfate and where the second coating comprises 90-99.0% (wt %) of methacrylic acid and ethyl acrylate copolymer, 0.3%-1% (wt %) of sodium lauryl sulfate and 1-3% (wt %) of polysorbate and additionally triethyl citrate.

Another embodiment of the invention is an oral dosage form where the capsule coating comprises as first coating 57.0% (wt %) of polyvinyl alcohol, 34.0% (wt %) of talc, 6% (wt %) of glycerol monocaprylate and 3% (wt %) of sodium lauryl sulfate and where the second coating comprises 97.0% (wt %) of methacrylic acid and ethyl acrylate copolymer, 0.7% (wt %) of sodium lauryl sulfate and 2.3% (wt %) of polysorbate and additionally triethyl citrate.

In another embodiment of the invention the triethyl citrate is present in an amount of 5-30% (wt %) of the second coating, or in an amount of 10-20% (wt %) of the second coating, preferably about 17% (wt %), most preferably 16.9% (wt %), of the second coating (8 mg per capsule).

In another embodiment of the invention there is additionally a third coating on top of the second coating. In another embodiment of the invention this coating is talc.

In further embodiments of the invention the talc is present in an amount of 0.1-3 mg per capsule, preferably 0.5-2 mg per capsule most preferably 1 mg per capsule.

Another embodiment of the invention is a method of producing an enteric-coated capsule containing a pharmaceutically active polypeptide which comprises applying to the capsule a first coating which comprises polyvinyl alcohol, talc, glycerol monocaprylate and sodium lauryl sulfate, and further applying a second coating on top of the first coating wherein the second coating comprises methacrylic acid and ethyl acrylate copolymer dispersion, sodium lauryl sulfate, polysorbate and triethyl citrate; in a further embodiment the method comprises applying a third coating on top of the second coating wherein the third coating is talc.

Another embodiment of the invention is an oral dosage form where the single coating comprises 80-99.0% (wt %), of methacrylic acid and ethyl acrylate copolymer, 0.1%-2% (wt %), of sodium lauryl sulfate and 0.5-4% (wt %), of polysorbate and additionally triethyl citrate.

Another embodiment of the invention is an oral dosage form where the single coating comprises 90-99.0% (wt %), of methacrylic acid and ethyl acrylate copolymer, 0.3%-1% (wt %), of sodium lauryl sulfate and 1-3% (wt %), of polysorbate and additionally triethyl citrate.

Another embodiment of the invention is an oral dosage form where the capsule coating comprises 97.0% (wt %), of methacrylic acid and ethyl acrylate copolymer, 0.7% (wt %), of sodium lauryl sulfate and 2.3% (wt %), of polysorbate and additionally triethyl citrate.

In another embodiment of the invention the triethyl citrate is present in an amount of 5-30% (wt %) of the coating, or in an amount of 10-20% of the coating, preferably about 17% (wt %) most preferably 16.9% (wt %) of the second coating (8 mg per capsule).

In another embodiment of the invention there is an additional coating on top of the single coating. In another embodiment of the invention this coating is talc.

In further embodiments of the invention the talc is present in an amount of 0.1-3 mg per capsule, preferably 0.5-2 mg per capsule most preferably 1 mg per capsule.

Another embodiment of the invention is a method of producing an enteric-coated capsule containing a pharmaceutically active polypeptide which comprises applying to the capsule a single coating wherein the coating comprises methacrylic acid and ethyl acrylate copolymer dispersion, sodium lauryl sulfate, polysorbate and triethyl citrate; in a further embodiment the method comprises applying an additional coating on top of the single coating wherein the additional coating is talc.

### Undercoatings of the Present Disclosure

| Type | Trademark | Polymer | Manufacturer |
|---|---|---|---|
| Water-soluble | Opadry ® AMB | Polyvinyl alcohol (PVA) | Colorcon |
| polymers | | | (Harleysville, PA, USA) |
| | Methocel ® E3/E5/E6/315 | Hydroxypropyl methyl cellulose | Dow Chemical (Midland, MI, USA) |
| | Walocel ® HM 3 PA/HM 5 PA/HM 6 | (HPMC) | Dow Wolff Cellulosics |
| | PA/HM 15 PA | | (Mitterland, MI, USA) |
| | Pharmacoat ® 603/606/615/645 | | Shin-Etsu (Tokyo, Japan) |
| | Sepifilm ® LP | | Seppic (Castres Cedex, France) |
| | Oxycellulose, Natrosol | Hydroxyethyl cellulose (HEC) | Ashland Aquaion (Covington, |
| | | | Kentucky, USA) |
| | Kollicoat ® IR Protect | Polyvinyl alcohol-polyethylene glycol | BASF (Ludwigshafen, Germany) |
| | | (PVA-PEG copolymer) | |
| | Kollicoat ® IR | Polyvinyl alcohol-polyethylene glycol | BASF (Ludwigshafen, Germany) |
| | AquaPolish ® | (PVA-PEG) | BioGrund |
| | Kollicoat ® Smartseal 30D | Methyl methacrylate and | BASF (Ludwigshafen, Germany) |
| | | diethylamino-ethyl ethacrylate | |
| | | copolymer dispersion | |
| | Klucel ™ | Hydroxypropyl cellulose (HPC) | Ashland (Covington, Kentucky, USA) |
| Insoluble | Kollicoat ® SR30 D | Polyvinyl acetate | BASF (Ludwigshafen, Germany) |
| polymers | Auqacoat ® | Ethyl cellulose | FMC (Philadelphia, PA, USA) |
| | Surelease ® (Fertigprodukt) | | Colorcon |
| | | | (Harleysville, PA, USA) |
| | Ethocel ™ | | Dow Chemical (Mitterland, MI, USA) |
| | Eastman CA | Cellulose acetate | Eastman (Rochester, MN, USA) |
| | Eudragit ® RL/ RS 30 D | Ammonio methacrylate | Evonik (Essen, Germany) |
| | Eudragit ® RL/ RS 12.5 | | |
| | Eudragit ® RL/ RS 100 | | |
| | Eudragit ® RL/ RS PO | | |
| | Aquapolish ® R | Ammonio methacrylate copolymer | Biogrund (Hünstetten, Germany) |
| | | (type A and type B) | |
| | Eudragit ® NE 30 D | Poly (ethyl acrylate-co-methyl | Evonik (Essen, Germany) |
| | Eudragit ® NM 30 D | methacrylate) 2:1 | |
| Entero-soluble polymers | SSB 55 Pharma | Shellac | Chineway (Stanghai, China) |
| | Aquacoat ® CPD | Cellulose acetate phthalate (CAP) | FMC (Philadelphia, PA, USA) |
| | Eastman C-A-P NF | | Eastman (Rochester, MN, USA) |
| | CAB Eastman | Cellulose acetate butyrate (CAB) | Eastman (Rochester, MN, USA) |
| | Eudragit ® L30D-55/ L 100-55 | Methyacrylic acid copolymer; Type A | Evonik (Essen, Germany) |
| | Eastacryl 30 D NF | | Eastman (Rochester, MN, USA) |
| | Kollicoat ® MAE 30 DP/100 P | | BASF (Ludwigshafen, Germany) |
| | Eudragit ® L 12.5/ L 100 | Methacrylic acid copolymer, Type B | Evonik (Essen, Germany) |
| | Eudragit ® S 12.5/S 100 | Methacrylic acid copolymer, Type C | Evonik (Essen, Germany) |
| | Eudragit ® FS 30 D | Methacrylic acid copolymer | Evonik (Essen, Germany) |
| | Kollicoat ® | Amino diethyl-methacrylate copolymer | BASF (Ludwigshafen, Germany) |
| | Smartseal 30 D | | |
| | Eudragit ® ®E/ E 12.5 | Amino dimethyl methacrylate | Evonik (Essen, Germany) |
| | Eudragit ® E PO | coploymer | |
| | Aquapolish ® E | Acrylic acid copolymer | Biogrund (Hünstetten, Germany) |
| | Keltone LV CR | Sodium alginate | FMC (Philadelphia, PA, USA) |
| | Akucell | Carboxymethyl cellulose CMC | Ashland Aqualon (Covington, |
| | | | Kentucky, USA) |

An additional embodiment is an oral dosage form comprising a capsule containing a formulation comprising a therapeutic agent wherein the capsule comprises a first coating comprising hydroxypropyl methylcellulose (HPMC) or hydroxypropyl cellulose (HPC) or hydroxyethylcellulose or shellac or cellulose acetate phthalate or cellulose acetate butyrate or sodium alginate or carboxymethylcellulose or polyvinylpyrrolidone and further comprises a second coating on top of the first coating comprising methacrylic acid and ethyl acrylate copolymer dispersion. There may be a third coating on top of the second coating e.g. talc.

The therapeutic agent may be a polypeptide, e.g., octreotide or terlipressin

The formulation may be as described herein e.g. may comprise a medium chain fatty acid salt and polyvinylpyrrolidone (PVP).

Another embodiment is an oral dosage form comprising a capsule or tablet or sachet containing pellets containing a therapeutic agent wherein the pellets (also termed microparticles and minitablets) comprise a first coating comprising polyvinyl alcohol, and further comprise a second coating on top of the first coating comprising methacrylic acid and ethyl acrylate copolymer dispersion (generally termed Eudragit L100). The therapeutic agent may be a polypeptide e.g. octreotide or terlipressin.

The formulation may be as described herein e.g. may comprise a medium chain fatty acid salt and polyvinylpyrrolidone (PVP).

Exemplary dosage forms include gelatin or vegetarian capsules like starch or hydroxypropylmethyl cellulose (“HPMC”) capsules, enteric coated, containing the bulk drug product. Capsules which may be used to encapsulate the compositions of this invention are known in the art and are described for example in Pharmaceutical Capsules edited by Podczech and Jones, Pharmaceutical Press (2004) and in Hard gelatin capsules today—and tomorrow, 2nd edition, Steggeman ed published by Capsugel Library (2002).

### Coatings—Examples

### OPADRY AmbII

OPADRY® AmbII is a brand name formulation (Colorcon Corporation) composed of PVA a plasticizer and an optional pigment. It is used mainly as a moisture barrier film coating and as a sub-coat to improve the adhesion of functional coatings onto the dosage surface.

Eudragit L 30 D-55 Eudragit is a brand name of Evonik industries (Germany). Kollicoat produces a similar product.
97.0% METHACRYLIC ACID COPOLYMER TYPE C (NF, PhEur,JPE)
0.7% Sodium lauryl sulfate Ph. Eur./NF
2.3% Polysorbate 80 Ph. Eur./NF

This product is a dispersion of copolymer that is produced using methacrylic acid and ethyl acrylate in an aqueous solution consisting of polysorbate 80 and sodium lauryl sulfate.

Chemical Name Dispersion of poly[(methacrylic acid)-co-(ethyl acrylate)]

### Structural Formula:

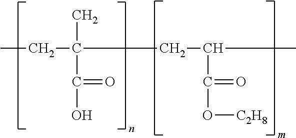

Coatings are applied as known in the art. See for example Aqueous Polymeric Coatings for Pharmaceutical Dosage Forms (2017) Linda A. Felton ed., CRC press

### Oral Dosage Form

The oral dosage forms described herein include a medicament in particular a polypeptide and a medium chain fatty acid salt in intimate contact or association with a substantially hydrophobic medium, encased in a capsule and coated with at least two different coatings to provide release of the contents between pH 4.5-6.0

The polypeptide of this invention is for example terlipressin and analogs (agonists) thereof, or octreotide and analogs (agonists) thereof.

The medicament may alternatively be a non-polypeptide

For example, the polypeptide and the medium chain fatty acid or derivative thereof may be coated, suspended, sprayed by or immersed in a substantially hydrophobic medium forming a suspension. The compositions of the invention are not emulsions. The compositions are oily suspensions and the amount of water in the compositions is very low, usually 1% or less or 0.5% or less. This is known as Chiasma's TPE® technology

The suspension may be a liquid suspension incorporating solid material, or a semi-solid suspension incorporating solid material (an ointment). Many of the compositions described herein comprise a suspension which comprises an admixture of a hydrophobic medium and a solid form wherein the solid form comprises a therapeutically effective amount of polypeptide and at least one salt of a medium chain fatty acid, and wherein the medium chain fatty acid salt is present in the composition in an amount of 10% or more by weight. The solid form may comprise a particle (e.g., consist essentially of particles, or consist of particles). The particle may be produced by methods known in the art e.g., by lyophilization, by spray drying by granulation or by roller compaction.

The medium chain fatty acid salt may generally facilitate or enhance permeability and/or absorption of polypeptide. In some embodiments, the medium chain fatty acid salts include derivatives of medium chain fatty acid salts. The polypeptide and the medium chain fatty acid salt are in solid form, for example, a solid particle such as a lyophilized particle, granulated particle, pellet or micro-sphere. In preferred embodiments, the polypeptide and the medium chain fatty acid salt are both in the same solid form, e.g., both in the same particle. In other embodiments, the polypeptide and the medium chain fatty acid salt may each be in a different solid form, e.g. each in a distinct particle.

Unlike emulsions, where water is an essential constituent of the formulation, the compositions described herein provide a solid form such as a particle containing polypeptide, which is then associated with the hydrophobic (oily) medium. The amount of water in the compositions is generally less than 3% by weight, usually less than about 2% or less than 1% by weight or about 0.5% by weight or less.

The compositions described herein are suspensions which comprise an admixture of a hydrophobic medium and a solid form wherein the solid form comprises a therapeutically effective amount of polypeptide and at least one salt of a medium chain fatty acid. The solid form may be a particle (e.g., consist essentially of particles, or consist of particles). The particle may be produced by lyophilization or by spray drying or by granulation or by roller compaction. The medium chain fatty acid salt is generally present in the compositions described herein in an amount of 10% or more by weight. In certain embodiments, the medium chain fatty acid salt is present in the composition in an amount of 10%-50%, preferably 11%-18% or about 11%-17% or 12%-16% or 12%-15% or 13%-16% or 13%-15% or 14%-16% or 14%-15% or 15%-16% or most preferably 15% or 16% by weight, and the medium chain fatty acid has a chain length from about 6 to about 14 carbon atoms preferably 8, 9 or 10 carbon atoms.

In some embodiments in the compositions described above, the solid form including polypeptide also includes a stabilizer (e.g., a stabilizer of protein structure). Stabilizers of protein structure are compounds that stabilize protein structure under aqueous or non-aqueous conditions or can reduce or prevent aggregation of polypeptide, for example during a drying process such as lyophilization or by roller compaction or by spray drying or other processing step. Stabilizers of structure can be polyanionic molecules, such as phytic acid, polyvalent ions such as Ca, Zn or Mg, saccharides such as a disaccharide (e.g., trehalose, maltose) or an oligo or polysaccharide such as dextrin or dextran, or a sugar alcohol such as mannitol, or an amino acid such as glycine, or polycationic molecules, such as spermine, or surfactants such as polyoxyethylene sorbitan monooleate (Tween 80) or pluronic acid. Uncharged polymers, such as mannitol, methyl cellulose and polyvinyl alcohol, are also suitable stabilizers.

Although polyvinylpyrrolidone (PVP) is known in the art as a stabilizer, in the compositions of the invention described herein, a PVP polymer, for example PVP-12, can serve to increase the effect of the permeability enhancer, e.g., in a synergistic manner; see co-assigned U.S. Pat. Nos. 8,329,198 and 9,566,246. Dextran and other matrix forming polymers may have a similar effect as PVP does.

In some embodiments, a bulking agent may be added, for example, mannitol or glycin.

In a particular embodiment of the compositions described herein the salt of the fatty acid is sodium octanoate and the hydrophobic medium is castor oil; in another particular embodiment the composition further comprises glyceryl monooleate and sorbitan monopalmitate or glyceryl monocaprylate and glyceryl tricaprylate and polyoxyethylene sorbitan monooleate; in another particular embodiment the composition further comprises glyceryl tributyrate, lecithin, ethylisovalerate and at least one stabilizer.

### Medium Chain Fatty Acid Salt:

The compositions described herein include the salt of a medium chain fatty acid or a derivative thereof in a solid form. For example, the salt of the medium chain fatty acid is in the form of a particle such as a solid particle. In some embodiments, the particle may be characterized as a granulated particle. In at least some embodiments, the solid form may generally result from a spray drying or evaporation process. In preferred embodiments, the salt of the medium chain fatty acid is in the same particle as the polypeptide. For example, the polypeptide and the salt of the medium chain fatty acid can be prepared together by first preparing a solution such as an aqueous solution comprising both polypeptide and the salt of the medium chain fatty acid and co-lyophilizing the solution to provide a solid form or particle that comprises both polypeptide and the salt of the medium chain fatty acid (and other ingredients). As described above, the resulting solid particles are associated with a hydrophobic medium. For example, the solid particles may be suspended or immersed in a hydrophobic medium.

In different embodiments of the compositions described herein the medium chain fatty acid salt may be in the same particle or in a different particle than that of the API. It is believed that if the medium chain fatty acid salt and polypeptide are dried after solubilization together in the hydrophilic fraction then they are in the same particle in the final powder.

Medium chain fatty acid salts include those having a carbon chain length of from about 6 to about 14 carbon atoms. Examples of fatty acid salts are sodium hexanoate, sodium heptanoate, sodium octanoate (also termed sodium caprylate), sodium nonanoate, sodium decanoate, sodium undecanoate, sodium dodecanoate, sodium tridecanoate, and sodium tetradecanoate. In some embodiments, the medium chain fatty acid salt contains a cation selected from the group consisting of potassium, lithium, ammonium and other monovalent cations e.g. the medium chain fatty acid salt is selected from lithium octanoate or potassium octanoate or arginine octanoate or other monovalent salts of the medium chain fatty acids.

In general, the amount of medium chain fatty acid salt in the compositions described herein may be from 10% up to about 50% by weight of the bulk pharmaceutical composition. For example, the medium chain fatty acid salt may be present at in amount of about 10%-50%, preferably about 11%-40% most preferably about 11%-28% by weight for example at about 12%-13%, 13%-14%, 14%-15%, 15%-16%, 16%-17%, 17%-18%, 18%-19%, 19%-20%, 20%-21%, 21%-22%, 2 2%-23%, 23%-24%, 2 4%-25%, 25%-26%, 26%-27%, or 27%-28% by weight of the bulk pharmaceutical composition. In other embodiments the medium chain fatty acid salt may be present in an amount of at least about 11%, at least about 12%, at least about 13%, at least about 14%, at least about 15% at least about 16%, at least about 17%, at least about 18%, at least about 19%, at least about 20%, at least about 21%, at least about 22%, at least about 23%, at least about 24%, at least about 25%, at least about 26%, at least about 27% or at least about 28% by weight of the bulk pharmaceutical composition. In specific embodiments, the medium chain fatty acid salt (sodium, potassium, lithium or ammonium salt or a mixture thereof) is present at about 12%-21% by weight of the bulk pharmaceutical composition preferably 11%-18% or about 11%-17% or 12%-16% or 12%-15% or 13%-16% or 13%-15% or 14%-16% or 14%-15% or 15%-16% or most preferably 15% or 16%. In specific embodiments, the medium chain fatty acid salt (having a carbon chain length of from about 6 to about 14 carbon atoms particularly 8, 9 or 10 carbon atoms) is present at about 12%-21% by weight of the bulk pharmaceutical composition preferably 11%-18% about 11%-17% or 12%-16% or 12%-15% or 13%-16% or 13%-15% or 14%-16% or 14%-15% or 15%-16% or most preferably 15% or 16%. In specific embodiments the medium chain fatty acid salt (for example salts of octanoic acid, salts of suberic acid, salts of geranic acid) is present at about 12%-21% by weight of the bulk pharmaceutical composition preferably 11%-18% about 11%-17% or 12%-16% or 12%-15% or 13%-16% or 13%-15% or 14%-16% or 14%-15% or 15%-16% or most preferably 15% or 16%. In certain embodiments, the medium chain fatty acid salt is present in the solid powder in an amount of 50% to 90%, preferably in an amount of 70% to 80%.

One embodiment of the invention comprises a composition comprising a suspension which consists essentially of an admixture of a hydrophobic medium and a solid form wherein the solid form comprises a therapeutically effective amount polypeptide and at least one salt of a medium chain fatty acid, and wherein the medium chain fatty acid salt is not a sodium salt. The salt may be the salt of another cation e.g. lithium, potassium or ammonium; an ammonium salt is preferred.

### Matrix Forming Polymer:

In certain embodiments the composition of the invention comprises a suspension which comprises an admixture of a hydrophobic medium and a solid form wherein the solid form comprises a therapeutically effective amount of polypeptide, at least one salt of a medium chain fatty acid and a matrix forming polymer, and wherein the matrix forming polymer is present in the composition in an amount of 3% or more by weight. In certain embodiments the composition comprises a suspension which consists essentially of an admixture of a hydrophobic medium and a solid form wherein the solid form comprises a therapeutically effective amount of polypeptide, at least one salt of a medium chain fatty acid and a matrix forming polymer, and wherein the matrix forming polymer is present in the composition in an amount of 3% or more by weight. In particular embodiments the matrix forming polymer is dextran or a polyvinylpyrrolidone polymer (PVP), obtainable in various molecular weights from BASF. In particular embodiments the polyvinylpyrrolidone is present in the composition in an amount of about 2% to about 20% by weight, preferably in an amount of about 3% to about 18% by weight, more preferably in an amount of about 5% to about 15% by weight, most preferably in an amount of about 10% by weight. In certain particular embodiments the polyvinylpyrrolidone is PVP-12 and/or has a molecular weight of about 3000. Other matrix forming polymers have a similar effect in the compositions of the invention; such matrix forming polymers include ionic polysaccharides (for example alginic acid and alginates) or neutral polysaccharides (for example dextran and HPMC), polyacrylic acid and poly methacrylic acid derivatives and high molecular weight organic alcohols (for example polyvinyl alcohol).

### Hydrophilic Fraction:

In embodiments of the invention, the above compounds, including polypeptide and the medium chain fatty acid salt are solubilized in an aqueous medium and then dried to produce a powder. The drying process may be achieved for example by lyophilization or spray drying or granulation or by roller compaction. The powder obtained is termed the “hydrophilic fraction”. In the hydrophilic fraction water is normally present in an amount of less than 6% or less than 3% or about 2% or less.

Lyophilization may be carried out by methods known in the art e.g. as described in Lyophilization: Introduction and Basic Principles, Thomas Jennings, published by Interpharm/CRC Press Ltd (1999, 2002) The lyophilizate may optionally be milled (e.g. below 150 micron) or ground in a mortar. During industrial production the lyophilizate is preferably milled before mixing of the hydrophilic fraction and the hydrophobic medium in order to produce batch-to-batch reproducibility.

Spray drying may be carried out by methods known in the art e.g. as described by Walters et al (2014) Next Generation Drying Technologies for Pharmaceutical Applications, J. of Pharm Sci 103; 2673-2695

Granulation may be carried out as shown by methods known in the art e.g. as described in Granulation, Salman et al, eds, Elsevier (2006) and in Handbook of Pharmaceutical Granulation Technology, 2^{nd} edition, (2005) Dilip M. Parikh, ed. Various binders may be used in the granulation process such as celluloses (including microcrystalline celluloses), lactoses (e.g., lactose monohydrate), dextroses, starch and mannitol and other binders as described in the previous two references.

### Hydrophobic Medium (Lipophiic Fraction):

Oil: As described above, in the compositions of the invention described herein the polypeptide and the medium chain fatty acid salt are in intimate contact or association with a hydrophobic medium. For example, one or both may be coated, suspended, immersed or otherwise in association with a hydrophobic medium. Suitable hydrophobic mediums can contain, for example, aliphatic, cyclic or aromatic molecules. Examples of a suitable aliphatic hydrophobic medium include, but are not limited to, mineral oil, fatty acid monoglycerides, diglycerides, triglycerides, ethers, esters, and combinations thereof. Examples of a suitable fatty acid are octanoic acid, decanoic acid and dodecanoic acid, also C7 and C9 fatty acids and di-acidic acids such as sebacic acid and suberic acid, and derivatives thereof. Examples of triglycerides include, but are not limited to, long chain triglycerides, medium chain triglycerides, and short chain triglycerides. For example, the long chain triglyceride can be castor oil or coconut oil or olive oil, and the short chain triglyceride can be glyceryl tributyrate and the medium chain triglyceride can be glyceryl tricaprylate. Monoglycerides are considered to be surfactants and are described below. Exemplary esters include ethyl isovalerate and butyl acetate. Examples of a suitable cyclic hydrophobic medium include, but are not limited to, terpenoids, cholesterol, cholesterol derivatives (e.g., cholesterol sulfate), and cholesterol esters of fatty acids. A non-limiting example of an aromatic hydrophobic medium includes benzyl benzoate.

In some embodiments of the compositions described herein, it is desirable that the hydrophobic medium include a plurality of hydrophobic molecules. In some embodiments of the compositions described herein the hydrophobic medium also includes one or more surfactants (see below).

In some embodiments of the compositions described herein, the hydrophobic medium also includes one or more adhesive polymers such as methylcellulose, ethylcellulose, hydroxypropylmethylcellulose (HPMC), or poly(acrylate) derivative Carbopol®934P (C934P). Such adhesive polymers may assist in the consolidation of the formulation and/or help its adherence to mucosal surfaces.

### Surface Active Agents (Surfactants):

The compositions of this invention described herein can further include a surface-active agent. For example, the surface-active agent can be a component of the hydrophobic medium as described above, and/or the surface-active agent can be a component of a solid form as described above, for example in the solid form or particle that includes the polypeptide.

Suitable surface-active agents include ionic and non-ionic surfactants. Examples of ionic surfactants are lecithin (phosphatidyl choline), bile salts and detergents. Examples of non-ionic surfactants include monoglycerides, cremophore, a polyethylene glycol fatty alcohol ether, a sorbitan fatty acid ester, a polyoxyethylene sorbitan fatty acid ester, Solutol HS15, or a poloxamer or a combination thereof. Examples of monoglycerides are glyceryl monocaprylate (also termed glyceryl monooctanoate), glyceryl monodecanoate, glyceryl monolaurate, glyceryl monomyristate, glyceryl monostearate, glyceryl monopalmitate, and glyceryl monooleate. Examples of sorbitan fatty acid esters include sorbitan monolaurate, sorbitan monooleate, and sorbitan monopalmitate (Span 40), or a combination thereof. Examples of polyoxyethylene sorbitan fatty acid esters include polyoxyethylene sorbitan monooleate (Tween 80), polyoxyethylene sorbitan monostearate, polyoxyethylene sorbitan monopalmitate or a combination thereof. The commercial preparations of monoglycerides that were used also contain various amounts of diglycerides and triglycerides.

Compositions described herein including a surface-active agent generally include less than about 12% by weight of total surface active agent (e.g., less than about 10%, less than about 8%, less than about 6%, less than about 4%, less than about 2%, or less than about 1%). In particular embodiments of the invention the total sum of all the surfactants is about 6%.

Methods of Making Pharmaceutical Compositions and the Compositions Produced

Also included in the invention are methods of producing the compositions described herein. Thus one embodiment of the invention is a process for producing a pharmaceutical composition which comprises preparing a water-soluble composition comprising a therapeutically effective amount of polypeptide and a medium chain fatty acid salt (as described above), drying the water soluble composition to obtain a solid powder, and suspending the solid powder in a hydrophobic medium, to produce a suspension containing in solid form polypeptide and the medium chain fatty acid salt, thereby producing the pharmaceutical composition, wherein the pharmaceutical composition contains 10% or more by weight of medium chain fatty acid salt.

One embodiment is a process for producing a pharmaceutical composition which comprises providing a solid powder of a therapeutically effective amount of polypeptide and a solid powder comprising a medium chain fatty acid salt, and suspending the solid powders in a hydrophobic medium, to produce a suspension containing in solid form the polypeptide and the medium chain fatty acid salt, thereby producing the pharmaceutical composition, wherein the pharmaceutical composition contains 10% or more by weight of medium chain fatty acid salt.

In one embodiment of the processes and compositions described herein, the water-soluble composition is an aqueous solution. In certain embodiments, the drying of the water-soluble composition is achieved by lyophilization (freeze-drying), or by spray-drying or by granulation or by roller compaction. In certain embodiments, the drying step removes sufficient water so that the water content in the bulk pharmaceutical composition is lower than about 6% by weight, about 5% by weight, about 4% by weight, about 3% or about 2% or about 1% or about 0.5% or less by weight. In certain embodiments of the processes and compositions described herein the drying step removes an amount of water so that the water content in the solid powder is lower than 6% or 5% or 4% or 3% or preferably lower than 2% by weight. The water content is normally low and the water may be adsorbed to the solid phase during lyophilization i.e. the water may be retained by intermolecular bonds. In certain embodiments, the water-soluble composition additionally comprises a stabilizer for example methyl cellulose. In preferred embodiments of the processes and compositions described herein the hydrophobic medium is castor oil or glyceryl tricaprylate or glyceryl tributyrate or a combination thereof and may additionally contain octanoic acid; in certain embodiments the hydrophobic medium comprises an aliphatic, olefinic, cyclic or aromatic compound, a mineral oil, a paraffin, a fatty acid such as octanoic acid, a monoglyceride, a diglyceride, a triglyceride, an ether or an ester, or a combination thereof. In certain embodiments of the processes and compositions described herein the triglyceride is a long chain triglyceride, a medium chain triglyceride preferably glyceryl tricaprylate or a short chain triglyceride preferably glyceryl tributyrate, and the long chain triglyceride is castor oil or coconut oil or a combination thereof. In certain embodiments of the processes and compositions described herein the hydrophobic medium comprises castor oil or glyceryl tricaprylate or glyceryl tributyrate or a combination or mixture thereof and may additionally comprise octanoic acid. In certain embodiments of the processes and compositions described herein the hydrophobic medium comprises glyceryl tricaprylate or a low molecular weight ester for example ethyl isovalerate or butyl acetate. In certain embodiments of the processes and compositions described herein the main component by weight of the hydrophobic medium is castor oil and may additionally comprise glyceryl tricaprylate. In certain embodiments of the processes and compositions described herein the main component by weight of the hydrophobic medium is glyceryl tricaprylate and may additionally comprise castor oil.

In certain embodiments, the composition comprises a suspension which consists essentially of an admixture of a hydrophobic medium and a solid form wherein the solid form comprises a therapeutically effective amount of polypeptide and at least one salt of a medium chain fatty acid, and wherein the medium chain fatty acid salt is present in the composition in an amount of 10% or more by weight. In certain embodiments, the hydrophobic medium consists essentially of castor oil, glyceryl monooleate and glyceryl tributyrate; or the hydrophobic medium consists essentially of glyceryl tricaprylate and glyceryl monocaprylate; or the hydrophobic medium consists essentially of castor oil, glyceryl tricaprylate and glyceryl monocaprylate. In certain embodiments, the hydrophobic medium comprises a triglyceride and a monoglyceride and in certain particular embodiments the monoglyceride has the same fatty acid radical as the triglyceride. In certain of these embodiments the triglyceride is glyceryl tricaprylate and the monoglyceride is glyceryl monocaprylate. In certain embodiments, the medium chain fatty acid salt in the water-soluble composition has the same fatty acid radical as the medium chain monoglyceride or as the medium chain triglyceride or a combination thereof. In certain of these embodiments the medium chain fatty acid salt is sodium caprylate (sodium octanoate) and the monoglyceride is glyceryl monocaprylate and the triglyceride is glyceryl tricaprylate.

Many of the compositions described herein comprise a suspension which comprises an admixture of a hydrophobic medium and a solid form wherein the solid form comprises a therapeutically effective amount of polypeptide and at least one salt of a medium chain fatty acid, and wherein the medium chain fatty acid salt is present in the composition preferably in an amount of 10% or more by weight. The solid form may be a particle (e.g., consist essentially of particles, or consists of particles). The particle may be produced by lyophilization or by granulation or by spray drying or by roller compaction. In one embodiment the formulation consists essentially of or comprises a suspension which comprises an admixture of a hydrophobic medium and a solid form wherein the solid form comprises a therapeutically effective amount of polypeptide and about 10-20% preferably 15% medium chain fatty acid salt preferably sodium octanoate and a polyvinylpyrrolidone polymer e.g. PVP-12; and wherein the hydrophobic medium comprises about 20-80%, preferably 30-70% medium or short chain triglyceride preferably glyceryl tricaprylate or glyceryl tributyrate, about 0-50% preferably 0-30% castor oil, about 3-10% surfactants, preferably about 6%, preferably glyceryl monocaprylate and Tween 80; in particular embodiments polypeptide is present in an amount of less than 33%, or less than 25%, or less than 10%, or less than 3% or less than 2%.

In the above formulations, the percentages are weight/weight.

In another embodiment the formulation comprises medium chain fatty acid salt and polyvinylpyrrolidone.

Under normal storage conditions, the polypeptide, within the formulations of the invention, is stable over an extended period of time. The chemical and physical state of the formulation is stable. Once administered to the intestine, the polypeptide is protected from damage by the GI environment since the formulations are oil-based and therefore a separate local environment is created in the intestine where terlipressin is contained in particles suspended in oil which confers stability in vivo.

In certain embodiments, the process produces a composition which consists essentially of polypeptide and a medium chain fatty acid salt and a hydrophobic medium. In embodiments of the invention the solid powder (solid form) consists essentially of polypeptide and a medium chain fatty acid salt. Further embodiments of the invention are pharmaceutical compositions produced by the process describe herein. The polypeptide and/or medium chain fatty acid salt, or any combination of polypeptide and other components, such as protein stabilizers, can be prepared in a solution of a mixture (e.g., forming an aqueous solution or mixture) which can be lyophilized together and then suspended in a hydrophobic medium. Other components of the composition can also be optionally lyophilized or added during reconstitution of the solid materials.

In some embodiments, the polypeptide is solubilized in a mixture, for example, including one or more additional components such as a medium chain fatty acid salt, a stabilizer and/or a surface-active agent, and the solvent is removed to provide a resulting solid powder (solid form), which is suspended in a hydrophobic medium. In some embodiments, polypeptide and/or the medium chain fatty acid salt may be formed into a granulated particle that is then associated with the hydrophobic medium (for example suspended in the hydrophobic medium or coated with the hydrophobic medium). If desired, the pharmaceutical composition may also contain minor amounts of non-toxic auxiliary substances such pH buffering agents, and other substances such as for example, sodium acetate and triethanolamine oleate.

In some embodiments, the solid form may be a particle (e.g., consist essentially of particles, or consists of particles). In some embodiments, the particle may be produced by lyophilization, by spray drying or by granulation or by roller compaction. In some embodiments of this process the fatty acid salt is sodium octanoate; in further embodiments of this process the medium chain fatty acid salt is present in the composition in an amount of about 11% to about 40% by weight or in an amount of about 11% to about 28% by weight or in an amount of about 15% by weight. In some embodiments of this process the composition additionally comprises a matrix forming polymer and in particular embodiments of this process the matrix forming polymer is dextran or a polyvinylpyrrolidone polymer (PVP); in further embodiments of this process the polyvinylpyrrolidone is present in the composition in an amount of about 2% to about 20% by weight or in an amount of about 4% to about 15% by weight, or in an amount of about 10% by weight. In certain embodiments of this process the polyvinylpyrrolidone polymer is PVP-12 and/or has a molecular weight of about 3000 Da. The composition may in addition include surfactants as described above. The solid form may also contain a binder. There also may be small quantities of other hydrophobic constituents as described above. The pharmaceutical products of these processes are further embodiments of the invention.

### Kits

Oral dosage forms may, if desired, be presented in a pack or dispenser device, such as an FDA approved kit, which may contain one or more unit dosage forms containing the active ingredient and instructions. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

### Definitions

As used herein, the term “polypeptide” refers to a molecule composed of covalently linked amino acids and the term includes peptides, polypeptides, proteins and peptidomimetics. A peptidomimetic is a compound containing non-peptidic structural elements that is capable of mimicking the biological action(s) of a natural parent peptide. Some of the classical peptide characteristics such as enzymatically scissile peptidic bonds are normally not present in a peptidomimetic. A peptidomimetic of a polypeptide may be an analog to that polypeptide.

The term “amino acid” refers to a molecule which comprises any one of the 20 naturally occurring amino acids or amino acids which have been chemically modified or synthetic amino acids.

As used herein, “analog” or agonist” of a polypeptide refers to a compound with similar chemical structure and biological activity to that peptide.

As used herein the term “pharmacologically or therapeutically effective amount” means that amount of a drug or pharmaceutical (therapeutic) agent (e.g. terlipressin, octreotide) that will elicit the biological or medical response of a tissue, system, animal or human that is being sought by a researcher or clinician and/or halts or reduces the progress of the condition being treated or which otherwise completely or partly cures or acts palliatively on the condition described herein or prevents development of the conditions described herein (e.g., acromegaly or hepatorenal syndrome, portal hypertension, varices eg bleeding esophageal varices, ascites and/or liver cirrhosis and/or cirrhotic ascites or severe cirrhotic ascites.

Administered “in combination”, as used herein, means that two (or more) different therapeutic agents are delivered to the subject during the course of the subject's affliction with the disorder, e.g., the two or more therapeutic agents are delivered after the subject has been diagnosed with the disorder and before the disorder has been cured or eliminated or treatment has ceased for other reasons. In some embodiments, the delivery of one therapeutic agent is still occurring when the delivery of the second begins, so that there is overlap in terms of administration. This is sometimes referred to herein as “simultaneous” or “concurrent delivery”. In other embodiments, the delivery of one therapeutic agent ends before the delivery of the other treatment begins. In some embodiments of either case, the therapeutic agents are more effective because of combined administration. For example, the second therapeutic agent is more effective, e.g., an equivalent effect is seen with less of the second therapeutic agent, or the second therapeutic agent reduces symptoms and side-effects to a greater extent, than would be seen if the second therapeutic agent were administered in the absence of the first therapeutic agent, or the analogous situation is seen with the first therapeutic agent. In some embodiments, delivery is such that the reduction in a symptom, or other parameter related to the disorder is greater than what would be observed with one therapeutic agent delivered in the absence of the other. The effect of the two therapeutic agents can be partially additive, wholly additive, or greater than additive. The delivery can be such that an effect of the first therapeutic agent delivered is still detectable when the second is delivered.

As used herein, the term “treatment” as for example in “method of treatment” or “treat” or “treating” refers to therapeutic treatment, wherein the object is to reduce or reverse or prevent the symptoms or side-effects of a disease or disorder. In some embodiments, the compounds or compositions disclosed herein are administered prior to onset of the disease or disorder. In some embodiments, the compounds or compositions disclosed herein are during or subsequent to the onset of the disease or disorder.

The function and advantages of these and other embodiments will be more fully understood from the following examples. These examples are intended to be illustrative in nature and are not to be considered as limiting the scope of the systems and methods discussed herein.

### EXAMPLES

### Example 1: Production of Mycapssa Capsules

The production of Mycapssa octreotide capsules has been described inter alia in co-assigned U.S. Pat. No. 8,329,198. The oily suspension (the capsule fill mass) contains, in addition to octreotide salt, 10% polyvinyl pyrrolidone K12, 15% sodium caprylate (octanoate), 0.6% magnesium chloride, 2% polysorbate 80 (Tween 80), 4% glyceryl monocaprylate and 65.1% glyceryl tricaprylate.

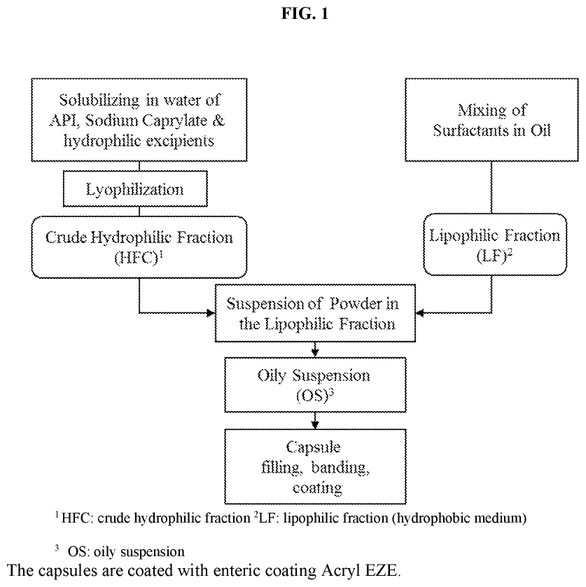

See FIG. 1 which depicts the manufacturing flow chart for preparation of octreotide capsules (Mycapssa® product).

### Example 2: Comparison of Different Coatings for 20 mg Octreotide Formulations and Dog PK Study Results

The current study evaluated the effect of altering the enteric coating and/or permeation enhancer on octreotide bioavailability. The beagle dog was used as the animal model, as it is an appropriate model for testing oral dosage forms. Various enteric coats and the permeation enhancer sodium caprate (NaC₁₀) were tested in place of sodium caprate (NaC₈) as used in Mycapssa. It was thought that NaC₁₀ may be more efficient in increasing macromolecule delivery across epithelial membranes.

This study thus compared the oral bioavailability and pharmacokinetic (PK) parameters after oral administration to dogs of Mycapssa and other formulation/coating prototypes. The various prototypes were tested in vitro in dissolution assays and then in vivo in dogs.

The coatings tested were as follows (more detail throughout the specification):

| Coating | |
|---|---|
| designation | Coating composition |
| NC02 | Eudragit/ Eudragit ® |
| NC03 | Eudragit FS30 ® |
| NC04 | White sub-coat (first coating) of OPADRY AmbII |
| | and a top coat (second coating) of Eudragit L 30 D-55 |
| NC05 | A mix of Eudragit FS30 and Eudragit L 30 D-55 |
| Mycapssa coating | Acryl-EZER ® |

The formulation changes were as follows:
1. Sodium caprate (NaC₁₀) as a permeation enhancer, instead of sodium caprylate (NaC₈)
2. Two concentrations of NaC₁₀ (15 and 20%)
3. Two enteric coats (NC02, NC04)—with potential different (higher) acid resistance than Acryl-EZE. Target—resistant up to pH 4.5 and dissolution above pH 5.5. The term resistant up to pH 4.5 means that each individual capsule has to exhibit below 10% dissolution or 0% dissolved up to 2 hours in citrate solution at pH 4.5 (and similarly for other pH values). The term dissolution above pH 5.5 means that the coating of the dosage form dissolves, and the dosage form releases its contents above pH 5.5 (and similarly for other pH values). NC02 is Eudragit/Eudragit. (This is called Eudragit/Eudragit because, just like with Acryl-EZE, the material is sprayed at 2 different rates to improve adhesion to the capsule. First a few mg are sprayed on at a slow rate and then the spray rate until is increased until the complete weight gain of coating is accomplished. The total amount of Eudragit on each capsule is the same in NC02 and NC04.

NC04 is comprises a white sub-coat of OPADRY ambII (first coating) and a Eudragit L 30 D-55 topcoat (second coating)
4. NC03. Enteric coat that dissolves above pH 7 comprising Eudragit FS30 which dissolves above pH-6.5 and was found not suitable for the intended purpose
5. NC05: A combination of two enteric polymers resulted in a coat that dissolved at around pH 6.5. This coating comprises of a mix of Eudragit FS30 and Eudragit L 30 D-55. This coating caused a long delay in the dissolution process and was found not suitable for the intended purpose.

The composition of the experimental capsules is shown below in Table 1.

TABLE 1
Composition of experimental capsules.

| Enhancer | | | | | | |
|---|---|---|---|---|---|---|
| | | | | | NaC₁₀ | NaC₁₀ |
| NaC₈ | NaC₈ | | | | (15%) | (20%) |
| Formulation code | | | | | | |
| F₁C₁P₁ | F₁C₂P₁ | F₁C₃P₁ | F₁C₄P₁ | F₁C₅P₁ | F₂C₂P₁ | F₃C₂P₁ |
| Batch No. | | | | | | |
| | | | OCT- | OCT- | | OCT- |
| | OCT-CCP- | OCT-CCP- | CCP- | CCP- | OCT-CCP- | CCP- |
| Mycapssa | 010 | 0012 | 011 | 016 | 013 | 014 |

HFC

| Octreotide | 20.0 | 20.0 | 20.0 |
|---|---|---|---|
| (free base) | | | |
| PVP-12 | 60.5 | 60.5 | 60.5 |
| MgCl₂ | 3.6 | 3.6 | 3.6 |
| Sodium | 90.8 | — | — |
| caprylate | | | |

| Sodium | — | 90.8 | 120.0 |
|---|---|---|---|
| caprate | | | |
| Total HFC | 174.9 | 174.9 | 204.1 |

LF

| GMC | 24.0 | 24.0 | 24.0 |
|---|---|---|---|
| GTC | 394.1 | 394.1 | 364.9 |
| Polysorbate | 12.0 | 12.0 | 12.0 |
| 80 | | | |
| Total weight | 605.0 | 605.0 | 605.0 |

Banding

| Gelatin | 9 | | | | | 9 | 9 |
|---|---|---|---|---|---|---|---|
| Polysorbate | 0.4 | | | | | 0.4 | 0.4 |
| 80 | | | | | | | |
| Coat code | Acryl | NC02 | NC04 | NC03 | NC05 | NC02 | NC02 |
| | EZE | | | | | | |
| | 93O18509 | | | | | | |

### L. Dissolution Tests

### Analytical Methods

Assay and impurities/degradation products for the octreotide capsule formulations were determined by a reverse-phase HPLC-UV/Fluo method through an Aeris peptide (or equivalent) column and acetonitrile:water: TFA mobile phase. Capsule contents were extracted with methanol, diluted and injected into the HPLC. The drug release (dissolution) profiles were determined using a USP-II apparatus with a two-stage dissolution method according to USP <711> and Ph. Eur. 2.9.3. Tests were conducted in 900 mL dissolution medium maintained at 37±0.5° C. at 50 rpm. The tests consisted of a two-hour acid stage dissolution in pH 4.5 citrate buffer (except for Mycapssa which is tested in 0.1N HCl, pH 1) followed by up to 60 minutes buffer stage dissolution in pH 6.8 phosphate buffer. For formulation F₁C₄P₁ a buffer at pH 7.2 (instead of 6.8) was used. The dissolution aliquots were withdrawn from the dissolution bath at the indicated time points and analyzed by HPLC.

### Finished Product Test Results

The assay/impurities and content uniformity (CU) results of the tested formulations appear below in Table 2. All the results were within the specifications.

TABLE 2
Assay and dissolution test of the tested capsules

| | | OCT-CCP- | OCT-CCP- | OCT-CCP- | OCT-CCP- | OCT-CCP- | OCT- |
|---|---|---|---|---|---|---|---|
| Name of Test | Specification | 010 | 011 | 012 | 013 | 014 | CCP-016 |
| Assay of | 90.0-110.0% | 102.7 | 102.7 | 102.7 | 95.7 | 98.6 | 102.7 |
| Octreotide | | | | | | | |
| CU | AV ≤ 15 | 15.4 | 15.4 | 15.4 | 6.9 | 15.3 | 15.4 |
| CU- Assay | — | 113.5 (1.4) | 113.5 (1.4) | 113.5 (1.4) | 104.1 (1.8) | 112.9 (1.7) | 113.5 (1.4) |
| (STDEV) | | | | | | | |

Dissolution Test

| | | | | | | | |
|---|---|---|---|---|---|---|---|
| Acid stage | <10% | 1 | 0 | 1 | 2 | 0 | 0 |
| (pH-4.5), 2 h | | | | | | | |
| Buffer stage | Q = 75% | 75 | 94 (120 min, | 101 | 97 | 95 | 93 (75 min) |
| (pH-6.8), | | | pH 7.2) | | | | |
| 45 min | | | | | | | |

### Dissolution Test Results

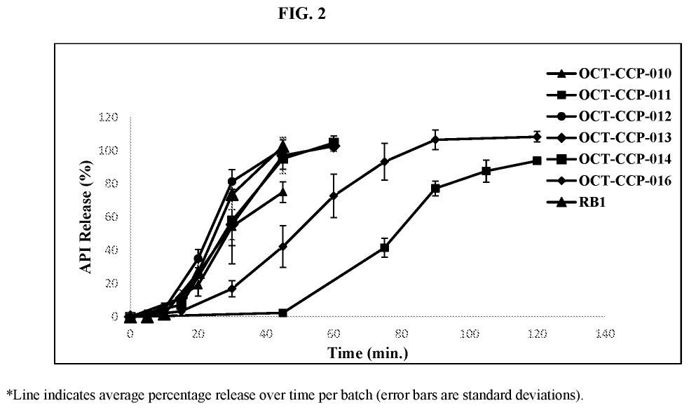

A two-stage (sequential) dissolution method was used. The acceptance criteria for the acidic stage was “no individual capsules dissolved above 10% in 2 hours”, and in the buffer stage a Q value of 75% at 45 minutes was set. Dissolution results of the prototypes at buffer stage (pH 6.8) are presented in FIG. 2 Different dissolution profiles were observed between the prototypes. Average data of API release (%) is presented.

In order to evaluate if the difference is significant, a statistical analysis was conducted. Results of the statistical analysis showed that no statistically significant differences are found between the slopes of batches where the confidence intervals overlap such as batch OCT-CCP-010 and OCT-CCP-013. Batch OCT-CCP-016 has a significantly slower rate of release compared with batches RB1 (Mycapssa), OCT-CCP-014, OCT-CCP-013, OCT-CCP-012 and OCT-CCP-010. Additionally, OCT-CCP-011 displays significantly even slower release than OCT-CCP-016. Note that all the batches except OCT-CCP-011 and OCT-CCP-016 are clustered together displaying faster release.

A series of 5 different coat formulations were tested, using capsules filled with the same TPE formulation as in the Mycapssa product (Phase 1 groups 1, 2, 3 and 4; Phase 2 group 4). The compositions of the various experimental coats resulted in an improved acid resistance compared to the coat of Mycapssa (Acryl-EZE coating). Mycapssa enteric coat was stable at pH 1, but peeled off the capsule in pH above 3 (a film floating in the dissolution vessel was observed). All the other experimental coats withstood 2 hours at pH 4.5 without opening. This higher acid resistance brought about lower Tlag and Tmax variabilities.

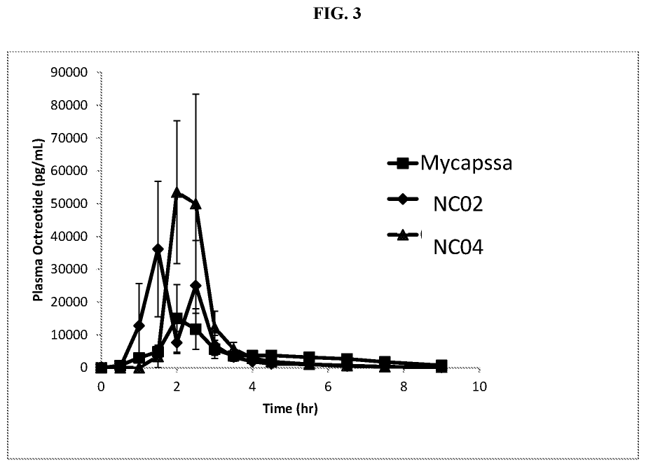

Note that some coats, namely NC02 and NC04, in combination with NaC8 as the permeation enhancer, produced a higher bioavailability than the Mycapssa product when tested in dogs. See below and FIG. 3 which presents dog PK results of NC02 and NC04 (the best two coats) against control (Mycapssa product).

Since the enteric film coats (NC02, NC04 and Mycapssa) dissolved at lower pH (below pH 5.5) except for NC03 and NC05, the dissolution rate of octreotide is not controlled by pH 6.8 and therefore cannot be predictive of the potential different rate of absorption of the different film coatings.

The experimental coats NC03 and NC05 were designed to dissolve at higher pH than NC02 and NC04. Thus, their dissolution profiles were distinctly different than the group of Mycapssa, NC02 and NC04: NC05 dissolved much slower at pH 6.8, while NC03 did not dissolve at all at pH 6.8, and its drug release profile at pH 7.2 is slower than all other coats. These differences in drug release profiles resulted in longer Tlag and Tmax, compared to NC02, NC03 and Mycapssa. Another effect was a lower bioavailability compared to NC02 and NC04.

### II. Animal Studies

### Experimental Design

Twenty-four male beagle dogs (8-12 kg) from an MPI Research stock colony of non-naïve beagle dogs were used after an acclimation period of 10 days. All animals were fasted for at least 12 hours prior to dosing and through the first 4 hours of blood sample collection. In treatments 1-4 in phase 1 and 2-4 in phase 2, in order to acidify the stomach of the dog, approximately 30 minutes prior to dosing each animal received a single subcutaneous (SC) injection of pentagastrin (0.12 mg/mL) at a dose level of 0.006 mg/kg and a dose volume of 0.05 mL/kg.

Each dog was used for 2 treatments (phase 1 and phase 2), with a wash-out period of 58 days. Blood was withdrawn at pre-determined times, processed into plasma, and frozen at −70° C. Octreotide concentration was measured using LC-MS/MS method. Octreotide exposure was calculated using the linear trapezoidal method. The octreotide SC data were used as reference for calculation of relative octreotide bioavailability (Frel) of orally administered formulations.

TABLE 3
Capsule description and study schedule (Phase1/Phase2)

| | | | | | | | |
|---|---|---|---|---|---|---|---|
| | | | | | Permeation | | |
| | | | Treatment | | enhancer per | | Target pH |
| | | | (Octreotide | | capsule | Enteric coat | of enteric coat |
| Group | Product code | Batch No. | dose) | Enhancer | mg (% W/W) | (name/code) | dissolving |

Phase 1

| | | | | | | | |
|---|---|---|---|---|---|---|---|
| 1 | CHIP4F₁C₁P₁ | *3573D15 | Oral | NaC₈ | 90 (15) | Acryl EZE | >1.2 |
| | (Mycapssa) | | capsule | | | | |
| | | | (20 mg) | | | | |
| 2 | CHIP4F₁C₂P₁ | OCT-CCP-010 | Oral | NaC₈ | 90 (15) | NC02 | >5.5 |
| | | | capsule | | | | |
| | | | (20 mg) | | | | |
| 3 | CHIP4F₁C₃P₁ | OCT-CCP-012 | Oral | NaC₈ | 90 (15) | NC04 | >5.5 |
| | | | capsule | | | | |
| | | | (20 mg) | | | | |
| 4 | CHIP4F₁C₄P₁ | OCT-CCP-011 | Oral | NaC₈ | 90 (15) | NC03 | 7.0 |
| | | | capsule | | | | |
| | | | (20 mg) | | | | |

Phase 2

| | | | | | | | |
|---|---|---|---|---|---|---|---|
| 1 | | | *SC; | — | — | — | — |
| | | | Sandostatin | | | | |
| | | | SC (0.1 mg) | | | | |
| 2 | CHIP4F₂C₂P₁ | OCT-CCP-013 | Oral | NaC₁₀ | 90 (15) | NC02 | >5.5 |
| | | | capsule | | | | |
| | | | (20 mg) | | | | |
| 3 | CHIP4F₃C₂P₁ | OCT-CCP-014 | Oral | NaC₁₀ | 120 (20) | NC02 | >5.5 |
| | | | capsule | | | | |
| | | | (20 mg) | | | | |
| 4 | CHIP4F₁C₅P₁ | OCT-CCP-016 | Oral | NaC₈ | 120 (20) | NC05 | >6.5 |
| | | | capsule | | | | |
| | | | (20 mg) | | | | |

*1 mL ampule containing octreotide (as acetate) at a concentration of 0.1 mg/mL (Sandostatin, Novartis Pharmaceuticals).

The compositions of the tested capsules are specified above in Table 1.

### Animal Study

The animal work was conducted at MPI Research (Kalamazoo, Michigan, USA). Non-naïve beagle dogs from an MPI Research stock colony (8-12 kg) were used as the animal model.

Test article administration: Animals were fasted for at least 12 hours prior to dosing and through the first 4 hours of blood sample collection (food was returned within 30 minutes following collection of the last blood sample at the 4 hour collection interval).

There were 58 days separating the 2 administration phases of Phase 1 and Phase 2.

Capsule Administration: The dog stomach pH is a good representative of the human fasted stomach with pentagastrin pretreatment. In order to acidify the stomach, approximately 30 minutes prior to dosing (±5 minutes) each animal received a single subcutaneous injection of pentagastrin (0.12 mg/mL) at a dose level of 0.006 mg/kg and a dose volume of 0.05 mL/kg. Details of pentagastrin preparation and use are known in the art. Designated animals received a single capsule dose of the appropriate test article formulation as outlined in Table 3.

Subcutaneous Administration: Designated animals in phase 2, group 1 received a single subcutaneous dose of Sandostatin 0.1 mg.

Blood collection: Blood samples (approximately 2 mL/sample) were collected from the jugular vein and placed into tubes containing K₃EDTA. Collection time points were: predose (0 hour) and at approximately 0.083, 0.25, 0.5, 0.75, 1, 1.33, 1.67, 2, 2.5 3, 4, and 5 hours postdose (13 blood samples) for the SC group, and predose (0 hour) and at approximately 0.5, 1, 1.5, 2, 2.5, 3, 3.5, 4, 4.5, 5.5, 6.5, 7.5, and 9 hours postdose (13 blood samples) for the oral delivered octreotide. All blood samples were placed on an ice block (or wet ice) following collection. The samples were centrifuged and the resulting plasma was separated and split into two approximately equal aliquots of 500 μl each. Each plasma sample was placed into pre-chilled tubes preloaded with 25 μL of aprotinin. The plasma samples were frozen at −60° C. to −90° C. in pre-labeled, plastic vials within 1 hour after centrifugation.

Bioanalysis: Analysis of plasma samples was performed using a LC-MS/MS method to determine octreotide in dog serum.

Pharmacokinetic analysis: Pharmacokinetic parameters for octreotide were calculated. The maximum plasma concentration (Cmax) and time to Cmax (Tmax) were taken directly from the data. The elimination rate constant, λz, and elimination half-life (t1/2) were calculated using standard methods.

Area under the curve was calculated and the absorption lag time, Tlag, was taken directly from the data as the first time after the first sampling time where the concentration was ≥LOQ. If the concentration was ≥LOQ at the first sampling time, then no lag time was estimated.

The bioavailability, F, of each oral treatment relative to the SC treatment was calculated as was the AUC(inf).

### Pharmacokinetics

The small number of animals per experimental group resulted in very high variability that makes it difficult to show statistically significant differences among treatments. Nevertheless, it is possible to point to a difference in Tlag and Tmax between Mycapssa and all the other experimental groups. The Tlag of Mycapssa was much shorter and variable (median of 0.51 hr and range of 0.50-3.48 hr) compared to the other experimental groups with Tlag medians in the range of 1.76-2-2.74, and smaller Tlag range within each group.

Compared to the control formulation (Mycapssa), for which the median Tmax was 1.76 hr, the medians were slightly longer for all groups, in the range of 2.02-3.50 hr. Formulations F₁C₄P₁ (OCT-CCP-011) had about 2-fold longer than Mycapssa −3.50 hr. As with the lag time, there was overlap among the ranges for the oral treatments

The mean values for Cmax, AUC(0-t), and AUC(inf) for Group 3 (F₁C₃P1=OCT-CCP-0012 termed also NC04) in phase 1 were the highest compared to the control or all the other oral formulations. Formulation Group 2, phase 1 (F₁C₂P1-OCT-CCP-010 termed also NC02) had results that were in between the control and NC04 formulation.

Group 4 in phase 1 (F₁C₄P1-OCT-CCP-011) had lower values for Cmax, AUC(0-t), and AUC(inf) than the control (Mycapssa).

Although the variability precludes a definitive ranking, based on AUC(0-t), the general rank order of bioavailability from the test and control formulations was F₁C₃P₁ ((NC04)>F₁C₂P₁>F₁C₁P₁ (Control)>F₁C₄P₁, F₁C₅P₁>F₃C₂P₁>F₂C₂P₁.

(Alternate codes: OCT-CCP-0012(NC04)>OCT-CCP-010(NC02)>Mycapssa control>OCT-CCP-011, OCT-CCP-016>OCT-CCP-014>OCT-CCP-013)

The best results were obtained for F₁C₃P1 (OCT-CCP-0012) which is the Mycapssa formulation plus capsules coated with NC04, a new coating which is OPADRY ambII White sub-coat (first coating) and a Eudragit L 30 D-55 top-coat (second coating). Also good bioavailability was obtained from capsules coated with NC02 (Eudragit L 30 D-55 only as coating) with the Mycapssa formulation. See FIG. 3

### Effect of Permeation Enhancer Type on Bioavailability

The permeation enhancer used in the Mycapssa product is sodium caprylate (NaC₈). In this study it was compared to 2 formulations (groups 2 and 3 in Phase 2) that included sodium caprate (NaC₁₀) as the permeation enhancer, at a level of 15% and 20% respectively. These experimental groups were coated with enteric coat NC02, and thus can be compared to Group 2 in Phase 1, that had an identical coat, but with NaC₈ as the permeation enhancer. The use of NaC₁₀ as the permeation enhancer reduced the bioavailability as compared to formulations that contained NaC₈ as the enhancer.

Note that two coats, namely NC02 and NC04, in combination with NaC₈ as the permeation enhancer, produced a higher bioavailability than Mycapssa when tested in dogs. See above and FIG. 3. This higher bioavailability could not have been predicted based on the dissolution profile in the buffer state (pH 6.8), as octreotide dissolution rate is not regulated by pH 6.8 and therefore there weren't many differences between the profiles of Mycapssa, NC02 and NC04 (Phase 1 groups 1, 2 and 3). The experimental coats NC03 and NC05 were designed to dissolve at higher pH than NC02 and NC04. Thus, their dissolution profiles were distinctly different than the group of Mycapssa, NC02 and NC04: NC05 dissolved much slower at pH 6.8, while NC03 did not dissolve at all at pH 6.8, and its drug release profile at pH 7.2 is slower than all other coats. These differences in drug release profiles resulted in longer Tlag and Tmax, compared to NC02, NC03 and Mycapssa. Another effect was a lower bioavailability compared to NC02 and NC04.

TABLE 4
Summary of PK parameters for capsules containing octreotide
with the two best coatings, compared to Mycapssa

| | Group 1 | Group 2 | Group 3 |
|---|---|---|---|
| | Mycapssa | NC02; C8 (15%) | NC04; C8 (15%) |
| Parameter* | CHIP4F₁C₁P₁ | CHIP4F₁C₂P₁ | CHIP4F₁C₃P₁ |
| Tlag (hr)† | 0.51 (6) | 1.76 (6) | 2.01 (6) |
| | [0.50-3.48] | [1.00-2.48] | [1.50-2.48] |
| Cmax (pg/mL) | 21,227 ± 24,673 (6) | 68,018 ± 39,456 (6) | 91,795 ± 74,297 (6) |
| Tmax (hr) | 1.76 (6) | 1.99 (6) | 2.02 (6) |
| | [1.02-5.48] | [1.00-2.50] | [1.50-2.48] |
| AUC(0-t) (hr × pg/mL) | 35,562 ± 24,267 (6) | 49,988 ± 25,875 (6) | 67,140 ± 48,347 (6) |
| AUC(inf) (hr × pg/mL) | 36,201 ± 29,112 (4) | 49,424 ± 28,905 (5) | 67,578 ± 48,392 (6) |
| λz (1/hr) | 0.5150 ± 0.1084 (4) | 0.5043 ± 0.1208 (5) | 0.5388 ± 0.2275 (6) |
| t½ (hr) | 1.40 ± 0.35 (4) | 1.47 ± 0.50 (5) | 1.53 ± 0.74 (6) |
| F (%)‡ | | | |
| Relative to SC | 1.38 | 1.89 | 2.59 |
| Relative to Mycapssa | 100.00 | 136.53 | 186.67 |

### Conclusions

This study evaluated the bioavailability of different enteric coatings that are more acid-stable at higher pH (up to pH 4.5). Although the high variability in absorption in the dogs precludes a definitive ranking, it can be said that the 6 test formulations had longer median values for lag time and Tmax compared to the control, although there was overlap among the ranges for all 7 oral formulations.

It was found that the bioavailability was higher in formulations with NaC₈ than NaC₁₀.

Based on AUC(O-t), the general rank order of bioavailability for the various enteric coats was NC04>NC02>Acryl-EZE (Mycapssa)>NC05>NC03. The bioavailability estimates for Group 3 (NC04) of phase 1 was substantially greater than the control (approximately two-fold increase in bioavailability, as compared to Mycapssa) and other treatment groups; NC02 had also significant increase in bioavailability, as compared to Mycapssa; see Table 4. Therefore, it is envisaged that both NC02 and NC04 coatings will be further evaluated for producing an oral dosage form with improved bioavailability, termed a second-generation capsule.

### Example 3: Production of 30 mg Octreotide Capsules with New Coating

The previous Example 2 describes octreotide second generation 20 mg capsules with a new improved coating (NC04) that is acid stable up to pH 4.5, which presented approximately two-fold improved bioavailability as compared to Mycapssa capsules.

Based on the coating of these 20 mg capsules it was decided to develop 30 mg capsules with the same coating (NC04) as described in this Example for the indications which need higher dosage of octreotide.

Five prototypes were manufactured at a dose of 30 mg as specified in Table 5 with the purpose of establishing a similar bioavailability as 20 mg capsules with NC04 coating.

TABLE 5
30 mg octreotide prototypes manufactured for dog PK study

| | | | |
|---|---|---|---|
| Product Code | Batch Number | Content details | Coating Code |

#Increase in Octreotide dosage strength

| | | | |
|---|---|---|---|
| CHIP4F₄C₃P₁ | OCT-CCP-035 | Mycapssa formulation “as is” | NC04* |
| | | with change in coating | |

Changes in HF

| | | | |
|---|---|---|---|
| CHIP4F₄C₃P₄ | OCT-CCP-037 | Sieved HF (400 μm) | NC04 |
| CHIP4F₄C₃P₂ | OCT-CCP-040 | Spray dried HF | NC04 |

Changes in LF

| | | | |
|---|---|---|---|
| CHIP4F₆C₃P₁ | OCT-CCP-039 | Croscarmellose in LF | NC04 |
| CHIP4F₅C₃P₁ | OCT-CCP-036 | less GTC | NC04 |

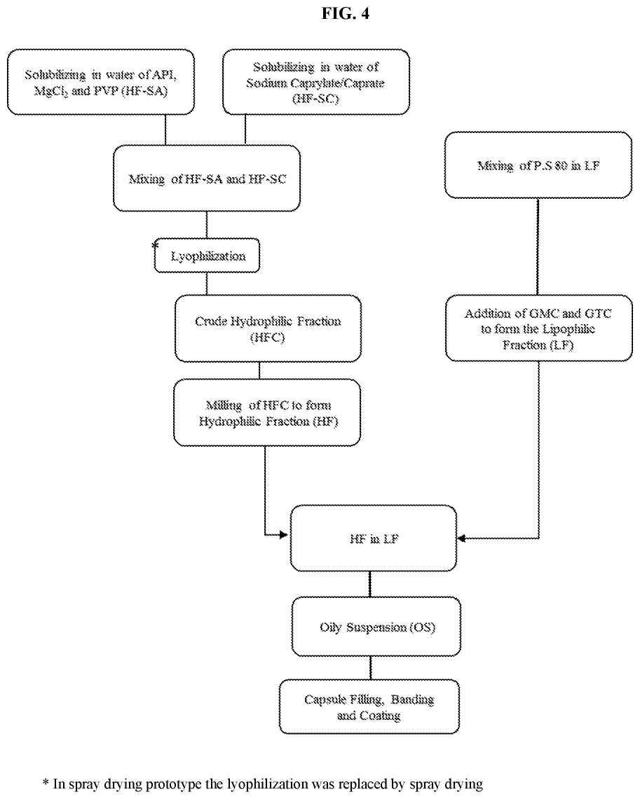

The manufacturing flow chart of production of 30 mg octreotide prototype capsules is shown in FIG. 4 and the composition of the HFC hydrophilic fraction is shown in Table 6 and the composition of the OS-oily suspension is shown in Table 7.

TABLE 6
Composition of HFC-hydrophilic fraction- (mg/capsule)

| | Batch number | | | | |
|---|---|---|---|---|---|
| | OCT-CCP- | OCT-CCP- | OCT-CCP- | OCT-CCP- | |
| | 035 | 036 | 037 | 039 | OCT-CCP- |
| | OCT-HFC-018 | | | | 040 |
| | OCT-HF- | OCT-HF- | OCT-HF- | OCT-HF- | Spray dried |
| Ingredient | 015 | 015 | 015 | 014 | HF-067#005B |
| Octreotide (free base) | 30.0 | | | | |
| PVP-12 | 60.0 | | | | |
| MgCl₂ | 3.6 | | | | |
| Sodium caprylate | 91.0 | | | | |
| Total HFC (hydrophilic | 184.6 | | | | |
| fraction) | | | | | |

TABLE 7
Composition of OS-oily suspension (per capsule)

| | Batch | | | | |
|---|---|---|---|---|---|
| | OCT-CCP- | OCT-CCP- | OCT-CCP- | OCT-CCP- | OCT-CCP- |
| Ingredient | 035 | 036 | 037 | 039 | 040 |
| number | OCT-OS-018 | OCT-OS-019 | OCT-OS-020 | OCT-OS-023 | OCT-OS-024 |
| HF | 188.7² | 184.6 | | | |
| Croscarmellose | NA | NA | NA | 24³ | NA |
| Sodium | | | | | |
| GMC | 24.0 | 24.0 | 24.0 | 24.0 | 24.0 |
| GTC | 375.3 | 189.7 | 379.4 | 355.4 | 379.4 |
| Polysorbate 80 | 12.0 | 12.0 | 12.0 | 12.0 | 12.0 |
| Total weight | 600.0 | 410.3 | 600.0 | 410.3 | 600.0 |

²Based on Octreotide acetate (34.1 mg)
³4% from 600 mg of OS

Filling of OS into Size 0 Hard Gelatin Capsules

Size 0 hard gelatin capsules (HGC) were manually filled with OS at a target fill weight of 600.0 mg±2.5%. Filling accuracy was confirmed at pre-determined intervals using an in-process weight check.

### Banding of Gelatin Capsules

Banding solution was prepared comprising gelatin powder (22%), polysorbate 80 (1.3%) and water for irrigation (76.7%) at a temperature of 50-55° C. After preparation of the banding solution, it was equilibrated at a low mixing speed in order to reduce the amount of air bubbles in the solution. Banding was than applied on the capsules at 43° C.±2° C. All banded capsules underwent a leak test under vacuum. Only capsules that have passed the leak test continued to the coating stage.

### The New Coating

The new coating formulation which gave highest bioavailability (designated NC04) consists of a three-layer film coat:

Sub-Coat (First Coating)—OPADRY ambII White

OPADRY ambII white is a polyvinyl alcohol (PVA) based, high performance moisture barrier film coating, originally developed for the coating of oral solid dosage forms that need to be protected from environmental moisture.

In these capsules OPADRY ambII white is employed as a sub-coat in order to optimize the adhesion of the enteric film to the hard gelatin capsules (HGC).

The results shown herein display an increased AUC using this sub-coat. Without being bound by theory, it is suggested that the PVA sub-coat serves as a moisture barrier, maintaining the necessary moisture level in the capsule shell. The purpose of the moisture barrier is to prevent the gelatin capsules shell drying out, i.e. losing their water, which must be at ˜13% for the capsules not to become brittle. Normally PVA is used to keep moisture out (particularly for tablets) but in the instant invention, without being bound by theory, PVA is being used to keep moisture in.

Enteric coat (2^{nd} coating)—Eudragit L 30 D-55 Eudragit L 30 D-55 is the aqueous dispersion of anionic polymers with methacrylic acid as a functional group. It is an effective and stable enteric coating.

### Topcoat (3^{rd} Coating)—Talc

Talc is employed as an anti-tacking agent, in order to prevent tackiness of the capsules during their storage in bulk.

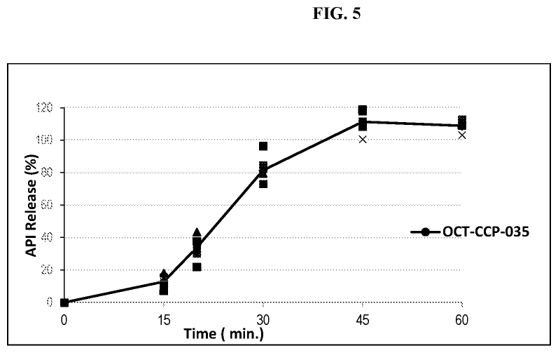

In vitro experiments were performed on the different capsules. Since the best BA results in dogs were obtained by capsule OCT-CCP-035, the in vitro results for this capsule are shown in FIG. 5.

FIG. 5 depicts dissolution of batch OCT-CCP-035 at pH 6.8. This shows that release at buffer stage (buffer phosphate, pH 6.8) conformed to specification (≥75% at 45 minutes). Average API release was 111% at 45 minutes. (Dissolution results higher than 100% were seen and this issue was investigated and found to be due to changes made in the sample preparation stage of the method which yielded higher than accurate peak areas.)

Dissolution results of batch OCT-CCP-035 at acidic stage pH 4.5 demonstrate acid resistivity of 6/6 capsules after 120 minutes. The new coating remains on the capsule at pH=4.5. In Mycapssa the coating comes off the capsule at pH=3.5 and the capsule dissolves.

Dissolution results of Mycapssa capsules at acidic stage (citrate buffer, pH 1.2) showed leakage from 1/6 capsules after 120 minutes (up to 3.2% API release). API Release was in accordance with the criterion (<10% at 120 minutes).

Dissolution test results of Mycapssa capsules at acidic stage (buffer citrate, pH 4.5) showed leakage from 6/6 capsules after 60 minutes. API Release was not in accordance with the criterion (<10% at 120 minutes).

Dissolution results of Mycapssa capsules at pH 6.8 (phosphate buffer, pH 6.8) showed API Release in accordance with the criterion (>75% at 45 minutes). Average API Release was 103% at 45 minutes. 2-stage dissolution=2 hours in acid media followed by 45 minutes in neutral buffer. For Mycapssa this is 2 hours in pH 1 acid media followed by 45 minutes in pH 6.8 buffer. For the 30 mg octreotide in new coating (NC04) it is 2 hours in pH 4.5 acid media followed by 45 minutes in pH 6.8 buffer. The dissolution of Mycapssa in buffer is the same as 30 mg octreotide in new coating (NC02) even though the acid exposure is different.

The relative bioavailabilities of the octreotide in the capsules when administered with pentagastrin to male Beagle dogs are compared in Table 8. Formulation CHIP4F₄C₃P₁ had the highest relative bioavailability, 125% and 124%, respectively, when given as 30 mg (Phase 1) and 60 mg (Phase 2) doses, respectively. Formulation CHIP4F₅C₃P₁, which had a lower amount of GTC, had the next highest bioavailability, 94%, and Formulations CHIP4F₄C₃P₄ and CHIP4F₄C₃P₂, which had sieved and spray dried HF, respectively, had the lowest bioavailabilities, 8000 and 71%, respectively.

TABLE 8
Summary of relative bioavailability for octreotide after
oral administration of 30 mg or 60 mg doses of CHIP4
capsules to male Beagle dogs - Phases 1 and 2.

| Phase | Group | Formulation | Batch No. | F(%)* |
|---|---|---|---|---|
| 1 | 1 | CHIP4F₄C₃P₁ | OCT-CCP-035 | 125 |
| | | | (“as is”) | |
| 2 | 2 | CHIP4F₄C₃P₁ | OCT-CCP-035 | 124 |
| | | | (“as is”) | |
| 1 | 2 | CHIP4F₅C₃P₁ | OCT-CCP-036 | 94 |
| | | | lower GTC | |
| 2 | 3 | CHIP4F₄C₃P₄ | OCT-CCP-037 | 80 |
| | | | sieved HF | |
| 2 | 1 | CHIP4F₄C₃P₂ | OCT-CCP-040 | 71 |
| | | | spray dried HF | |

*Calculated from the dose-corrected mean AUC(inf) relative to that for the control treatment (Mycapssa).

### Example 4: Production and Testing of Terlipressin Capsules

Terlipressin (also known as triglycyl lysine vasopressin) is a synthetic analogue of the neuropeptide hormone vasopressin and a pro-drug of 8-lysine vasopressin (LVP). Terlipressin is administered by an intravenous (iv) infusion or bolus at the hospital to treat acute bleeding of esophageal varices that develop in people who suffer from portal hypertension due to liver cirrhosis and is also given for hepatorenal syndrome types 1 and 2 and for ascites which may be cirrhotic ascites or severe cirrhotic ascites.

An oral terlipressin (TP) capsule is presented based on the formulation developed for 30 mg octreotide NC04 (Example 3). The current study evaluated the pharmacokinetic parameters in dogs of terlipressin given by IV bolus injection (2 escalating doses) and oral capsules (2 escalating doses). This Example describes the manufacturing and characteristics of terlipressin capsule prototypes used for a dog PK study and the results of that study.

Three prototypes were manufactured differing in their dosage strength (2 mg or 10 mg) and the quantity and type of PVP used (PVP-12 or PVP K-30), with the amount of PVP in the formulation adjusted to result in the same viscosity of the total formulation). The amounts of the other constituents (sodium caprylate, magnesium chloride, polysorbate 80, glyceryl monocaprylate and glyceryl tricaprylate) were essentially the same as for Mycapssa (Example 1).

### Manufacturing Process

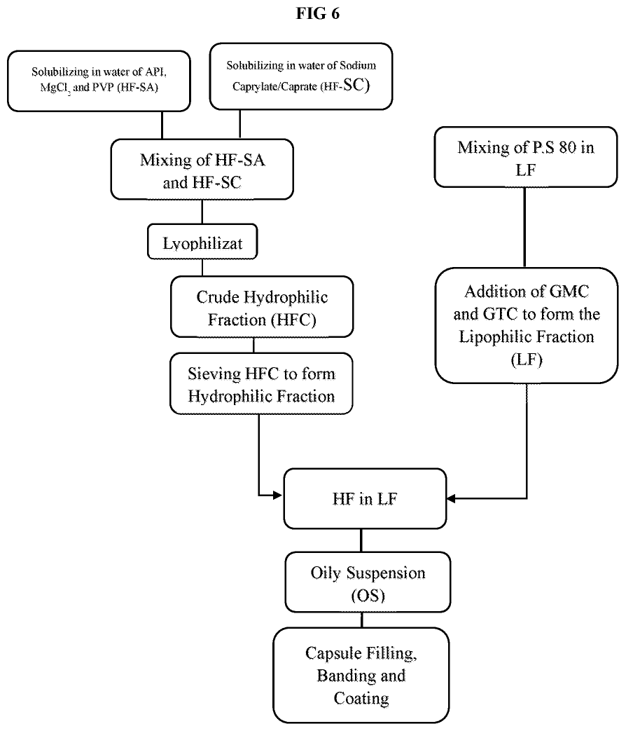

The manufacturing flow chart of terlipressin capsules is depicted in FIG. 6

### Preparation of Crude Hydrophilic Fraction and Hydrophilic Fraction (HF—Also Termed Hydrophobic Medium)

Terlipressin acetate, PVP-12 or PVP-30 and MgCl2 were dissolved in water to form the HF-SA solution. Separately, sodium caprylate was dissolved in water to form HF-SC solution.

After achieving clear solutions, the HF-SA and HF-SC solutions were combined (HF-SA was added into HF-SC) while mixing until an off-white suspension was achieved. The suspension was then lyophilized. After lyophilization, the dried HFC was sieved through a 100 mesh (150 μm) screen to form the HF.

### Preparation of Lipophilic Fraction (LF)

Glycerol monocaprylate (GMC) was melted in an oven at 55° C. The LF was prepared by mixing GMC with polysorbate 80 and glycerol tricaprylate (GTC) in a glass beaker at room temperature.

### Preparation of Oily Suspension (OS)

The HF was added to the LF while continuously stirred, followed by high shear mixing.

Filling of OS into Size 0 Hard Gelatin Capsules

Size 0 hard gelatin capsules were manually filled with OS using an Eppendorf pipette at a target fill weight of 600.0 mg±2.5%.

### Banding of Gelatin Capsules

The banding solution was prepared comprising gelatin powder (22%), polysorbate 80 (1.3%) and water for irrigation (76.7%) at a temperature of 50-55° C. After preparation of the banding solution, it was equilibrated at a low mixing speed in order to reduce the amount of air bubbles in the solution. Banding was than applied on the capsules at 43° C.±2° C. All banded capsules underwent a leak test. Only capsules that passed the leak test continued to the coating stages.

### Preparation of Coating Prototypes

Three coating layers were applied for each prototype. OPADRY ambII White was applied as a sub-coat (first coating) followed by Eudragit L 30 D-55 as a top coat (second coating) and talc as a third-coat (third coating). Dry coating formulations for these three batches are presented in Table 9. The coating in Table 9 is the NC04 coating.

Note that alternatively terlipressin capsules can be prepared as described above but with the Eudragit—only coating (NC02). The capsules are manufactured as described above and the coating is applied as described above but without the OPADRY amb II White coating (first coating).

TABLE 9
Dry coating formulation of coating prototype

| | | | | | | |
|---|---|---|---|---|---|---|
| | | | Theoretical | | | |
| | | | Quantity of | Capsule | | Actual |
| | | | Dry | surface | Actual | Weight |
| Batch | Material | Concentration | substances | area | Quantity | Gain |
| number | name | of solid film | (mg/cm²) | (cm²/cap) | of | (mg/cap) |

Sub coat

| | | | | | | |
|---|---|---|---|---|---|---|
| TP-CCP- | OPADRY | 100.00 | 5.00 | 5.00 | 5.82 | 29.10 |
| 001 | amb II | | | | | |
| TP-CCP- | white | | | 5.00 | 5.76 | 28.80 |
| 002 | | | | | | |
| TP-CCP- | | | | 5.00 | 5.48 | 27.40 |
| 003 | | | | | | |

Enteric coat

| | | | | | | |
|---|---|---|---|---|---|---|
| TP-CCP- | Eudragit | 83.13 | 7.84 | 5.00 | 7.96 | 39.82 |
| 001 | L 30 D-55 | | | | | |
| | TEC | 16.87 | 1.59 | 5.00 | 1.62 | 8.08 |
| | Total | 100.00 | 9.43 | 5.00 | 9.58 | 47.90 |
| TP-CCP- | Eudragit | 83.13 | 7.84 | 5.00 | 7.53 | 37.66 |
| 002 | L 30 D-55 | | | | | |
| | TEC | 16.87 | 1.59 | 5.00 | 1.53 | 7.64 |
| | Total | 100.00 | 9.43 | 5.00 | 9.06 | 45.30 |
| TP-CCP- | Eudragit | 83.13 | 7.84 | 5.00 | 8.65 | 43.23 |
| 003 | L 30 D-55 | | | | | |
| | TEC | 16.87 | 1.59 | 5.00 | 1.75 | 8.77 |
| | Total | 100.00 | 9.43 | 5.00 | 10.40 | 52.00 |

Top coat

| | | | | | | |
|---|---|---|---|---|---|---|
| TP-CCP- | Talc | 100.00 | 0.20 | 5.00 | 0.04 | 0.20 |
| 001 | | | | | | |
| TP-CCP- | | 100.00 | 0.20 | 5.00 | 0.04 | 0.20 |
| 002 | | | | | | |
| TP-CCP- | | 100.00 | 0.20 | 5.00 | −0.16 | −0.80 |
| 003 | | | | | | |

Finished product tests results of prototypes for the PK study are presented in Table 10 and the composition of the capsules produced is presented in Table 11.

TABLE 10
Finished product tests results of drug product prototypes

| Test | TP-CCP-001 | TP-CCP-002 | TP-CCP-003 |
|---|---|---|---|
| Appearance | White coated | White coated | White coated |
| | capsules | capsules | capsules |
| Assay of Terlipressin | 99.0 | 108.8 | 99.0 |
| (%) | | | |
| CU | 5.0 | 11.5 | 3.8 |
| CU- Assay | 99.8 (2.1) | 109.4 (1.5) | 98.8 (1.6) |
| (%)(STDEV) | | | |
| Related Substances: | | | |
| Terlipressin-COOH- | ≤0.1% | ≤0.1% | ≤0.1% |
| Total | ND* | ≤0.1% | ND* |

Dissolution Test

| | | | |
|---|---|---|---|
| Acid stage (pH-4.5), 2 h | 0 | 1 cap (40%) | 0 |
| Buffer stage (pH-6.8), | 87 | N.A | 72 |
| 45 min | | | |

*ND—Not Detected

TABLE 11
Composition of capsules produced (mg per capsule)

Batch Number

| | | |
|---|---|---|
| TP-CCP-001 | TP-CCP-002 | TP-CCP-003 |

Product code

| | | |
|---|---|---|
| TP-03 | TP-02 | TP-01 |

HF

| | | | |
|---|---|---|---|
| Terlipressin (free base) | 10.0 | 10.0 | 2.0 |
| PVP-K12 | — | 60.0 | 60.0 |
| PVP-K30 | 24.0 | — | — |
| MgCl₂ | 3.6 | 3.6 | 3.6 |
| Sodium caprylate | 91.0 | 91.0 | 91.0 |
| Total HFC | 128.6 | 164.6 | 156.6 |

OS

| | | | |
|---|---|---|---|
| Polysorbate 80 | 12.0 | 12.0 | 12.0 |
| Glycerol monocaprylate (GMC) | 24.0 | 24.0 | 24.0 |
| Glycerol tricaprylate (GTC) | 435.4 | 399.4 | 407.4 |
| Total | 600.0 | 600.0 | 600.0 |

Banding

| | | | |
|---|---|---|---|
| Gelatin | 9.0 | 9.0 | 9.0 |
| Polysorbate 80 | 0.4 | 0.4 | 0.4 |
| Coating code | NC04 | NC04 | NC04 |

NC04 is the new coating described in Table 9.

### Dissolution Test Results

A two-stage (sequential) dissolution method was used. The acceptance criteria for the acidic stage was “no individual capsules dissolved above 10% in 2 hours”, and in the buffer stage a Q value of 75% at 45 minutes was set.

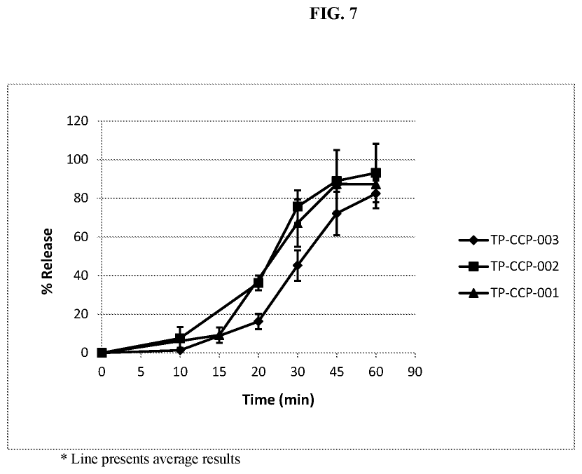

The dissolution results at pH 6.8 of the terlipressin prototypes used in the PK study are presented in FIG. 7.

Vessel 2 in the TP-CCP-002 batch showed outlier results (2440% release in 30 min) and therefore was excluded from the dissolution profile results. The capsule placed in vessel 5 failed to present acid resistivity (40.8% release at 120 min) and therefore was also excluded from the dissolution profile results.

### Dissolution Results:

Dissolution results of batch TP-CCP-001 at acidic stage (pH 4.5) demonstrated acid resistivity at 6/6 capsules after 120 minutes.

Dissolution results of batch TP-CCP-001 at pH 6.8 demonstrated API release in buffer stage (buffer phosphate, pH 6.8) in accordance with the criterion (≥75% at 45 minutes). Average API Release was 87% at 45 minutes.

Dissolution results of batch TP-CCP-002 at acidic stage (pH 4.5) demonstrated acid resistivity of 5/6 capsules after 120 minutes.

Dissolution results of batch TP-CCP-002 at pH 6.8 (phosphate buffer) demonstrated API release in accordance with the criterion (>75% at 45 minutes). Average API release was 96% at 45 minutes.

Dissolution results of batch TP-CCP-003 acidic stage (pH 4.5) demonstrated acid resistivity of 6/6 capsules after 120 minutes.

Dissolution results of batch TP-CCP-003 at pH 6.8 (phosphate buffer) demonstrated release lower than expected for modified release product according to ICH guideline (≥75% at 45 minutes). Average API release was 72% at 45 minutes.

### Dog PK Study Using Terlipressin Capsules

A dog PK study with terlipressin capsules was carried out essentially as described in Example 2. The key differences are as follows:

Test article administration: Animals were fasted for at least 12 hours prior to dosing and through the first 4 hours of blood sample collection (food was returned within 30 minutes following collection of the last blood sample at the 4 hour collection interval).

Animal dosing was performed as Phase land Phase 2. There were 7 days separating the 2 administration phases.

Intravenous administration: Animals in Phase 1 (groups 1 and 2) received a single iv of glypressin of 0.04 or 0.2 mg terlipressin base (0.24 or 1.2 mL/animal, respectively). Dosing was performed by 1 minute slow injection via the cephalic vein, using a catheter.

Capsule Administration: In order to acidify the stomach, approximately 30 minutes prior to dosing (±5 minutes) each animal received in a single subcutaneous injection of pentagastrin (0.12 mg/mL) at a dose level of 0.006 mg/kg and a dose volume of 0.05 mL/kg. Designated animals received one or two capsules of the appropriate test article formulation as outlined above.

Blood collection: Blood samples (approximately 3 mL/sample) were collected from the jugular vein in the following time points:

IV: predose, 0.083, 0.166, 0.33, 0.66, 1, 1.5, 2, 3, 4, 5, 6, and 9 hours post-dose.

Oral capsules: Predose, 0.5, 1, 1.5, 2, 2.5, 3, 3.5, 4, 4.5, 5.5, 6.5, 7.5, 9 and 12 hours post-dose.

Blood samples were placed into tubes containing K₂EDTA. The samples were centrifuged under refrigerated (2 to 8° C.) conditions within 30 minutes of sample collection and the resulting plasma was separated and split into two approximately equal aliquots. Each plasma sample was placed into pre-chilled tubes that were placed on dry ice until frozen at −60° to −90° C. Bioanalysis was performed for plasma concentrations of both terlipressin and LVP.

Bioanalysis of plasma samples was performed using a LC-MS/MS method for quantitation of terlipressin and [Lys8]—Vasopressin in Dog Plasma.

Pharmacokinetic analysis was performed essentially as described in Example 2.

### Results:

### Phase 1.

TABLE 12
Summary of PK parameters for terlipressin and lysine vasopressin after IV administration
of 0.04 mg and 0.2 mg doses of terlipressin to male Beagle dogs - Phase 1.

| | Terlipressin | | Lysine Vasopressin | |
|---|---|---|---|---|
| | Group 1 | Group 2 | Group 1 | Group 2 |
| Parameter* | 0.04 mg | 0.2 mg | 0.04 mg | 0.2 mg |
| Cmax (pg/mL) | 12,550 ± 782 (6) | 59,130 ± 30,875 (6) | 328 ± 37.5 (6) | 1,286 ± 599 (6) |
| Tmax (hr) | 0.083 (6) | 0.067 (6) | 0.67 (6) | 0.67 (6) |
| | [0.067-0.083] | [0.067-0.083] | [0.33-0.67] | [0.33-1.50] |
| AUC(0-t) (hr × pg/mL) | 1,820 ± 180 (6) | 9,133 ± 4,282 (6) | 452 ± 62.8 (6) | 1,693 ± 579 (6) |
| AUC(inf) (hr × pg/mL) | 1,772 ± 143 (5) | 9,138 ± 4,280 (6) | 455 ± 61.7 (6) | 1,696 ± 579 (6) |
| λz (1/hr) | 9.38 ± 0.37 (5) | 3.01 ± 0.31 (6) | 1.06 ± 0.07 (6) | 1.04 ± 0.14 (6) |
| t_{1/2} (hr) | 0.074 ± 0.003 (5) | 0.232 ± 0.022 (6) | 0.65 ± 0.04 (6) | 0.68 ± 0.11 (6) |

*Arithmetic mean ± standard deviation (N) except Tmax for which the median (N) is reported

As shown in Table 12, after IV administration of terlipressin at doses of 0.04 mg and 0.2 mg, there was a dose-related increase in the arithmetic mean plasma concentrations of terlipressin. The arithmetic mean values for Cmax, AUC(0-t), and AUC(inf) increased about 5-fold, consistent with the 5-fold higher dose. Plasma concentrations could be followed through 0.67 hr at the low dose and 1.5 hr at the high dose. Consequently, the data after the 0.2 mg dose should be considered more representative of the PK of terlipressin in the beagle dog. The arithmetic mean t %/2 was 0.074 hr for the 0.04 mg cohort and 0.232 hr for the 0.2 mg cohort (Table 10C). The difference is most likely due to the longer period of time that concentrations were ≥LOQ for the higher dose group.

The arithmetic mean plasma concentrations of LVP also increased in a dose-related manner although the increases in Cmax (3.9-fold) and both AUCs (3.7-fold) were less than the 5-fold increase in dose. The arithmetic mean t1/2 was essentially the same for both doses, 0.65 hr and 0.68 hr, 0.04 and 0.2 mg, respectively.

### Phase 2

TABLE 13
Summary of PK parameters for plasma terlipressin and LVP after oral administration of capsules to beagle dogs - Phase 2

| | Terlipressin | | | Lysine Vasopressin | | |
|---|---|---|---|---|---|---|
| | Group 1 | Group 2 | Group 3 | Group 1 | Group 2 | Group 3 |
| Parameter* | 0.04 mg | 0.2 mg | 0.2 mg | 0.04 mg | 0.2 mg | 0.2 mg |
| Tlag (hr) | ‡ | 2.00 (4) | 1.60 (3) | 2.00 (2) | 1.50 (6) | 1.60 (6) |
| | | [1.00-2.00] | [1.42-2.10] | [2.00-2.00] | [1.00-2.45] | [1.42-2.10] |
| Cmax (pg/mL) | ‡ | 22,705 ± 22,255 (4) | 11,567 ± 6,924 (3) | 29.0 ± 13.4 (2) | 2,921 ± 2,984 (6) | 527 ± 617 (6) |
| Tmax (hr) | ‡ | 2.25 (4) | 2.10 (3) | 2.00 (2) | 2.23 (6) | 2.10 (6) |
| | | [2.00-2.52] | [1.42-2.10] | [2.00-2.00] | [2.00-2.52] | [1.42-2.10] |
| AUC(0-t) (hr × pg/mL) | ‡ | 13,319 ± 11,282 (4) | 6,430 ± 4,269 (3) | 28.8 ± 14.2 (2) | 3,499 ± 3,757 (6) | 652 ± 766 (6) |
| AUC(inf) (hr × pg/mL) | ‡ | 13,334 ± 11,284 (4) | 6,451 ± 4,283 (3) | 31.1 ± 14.0 (2) | 3,504 ± 3,759 (6) | 655 ± 766 (6) |
| λz (1/hr) | ‡ | 1.27 ± 0.47 (4) | 0.80 ± 0.41 (3) | 1.27 ± 0.03 (2) | 1.02 ± 0.49 (6) | 1.35 ± 0.10 (6) |
| t½ (hr) | ‡ | 0.61 ± 0.24 (4) | 1.06 ± 0.62 (3) | 0.55 ± 0.012 (2) | 0.84 ± 0.41 (6) | 0.52 ± 0.04 (6) |
| F (%)† | ‡ | 1.46 | 0.71 | 0.18 | 2.07 | 0.39 |

As shown in Table 13, after oral administration of 2 mg capsules (Group 1), plasma terlipressin could not be detected in many dogs and in those for which it was, the number of concentrations ≥LOQ was too few (≤3) to do any PK analysis.

Both groups 2 and 3 received two terlipressin 10 mg capsules. The only difference between the 2 capsule formulations was the use of PVP-12 in formulation TP-02, and PVP-K30 in formulation TP-03. The arithmetic mean plasma terlipressin concentrations for Group 2 (TP-02), were higher than those for Group 3 (TP-03). Animals in Group 2 had an approximate 2-fold higher arithmetic mean Cmax, AUC(0-t), and AUC(inf) than did those in Group 3. The median and range for Tlag and Tmax were comparable for both 2 times 10 mg dosings. Based on AUC(inf) for these groups and that for Group 2 in Phase 1 (0.2 mg IV), the bioavailability was higher for group 2 than for group 3.

Plasma LVP concentrations were detectable for all 3 groups although the arithmetic mean concentrations after the 2 mg dose (Group 1) were substantially lower than for the 2 groups administered 20 mg (Group 2—TP-02; Group 3—TP-03). The increases between 2 mg and either 20 mg dosing in the arithmetic mean Cmax, AUC(0-t), and AUC(inf) were considerably greater than dose proportional. The median and range for Tlag and Tmax were comparable for both 20 mg dosings. Although the dose was the same, 20 mg, the exposure to LVP from the TP-02 formulation was >5-fold higher than from the TP-03 formulation.

The bioavailability was highest for TP-02, followed by TP-03 and then TP-01.

### Conclusions

The sensitivity of the bioanalytical methods for terlipressin and LVP appears to be suitable for the determination of plasma concentrations after IV and oral doses as low as 0.04 mg and 20 mg, respectively.

The PK of terlipressin and LVP appear to be linear after IV administration of 0.04 and 0.2 mg doses.

Plasma concentrations of terlipressin after oral administration of 2 mg were <LOQ in the majority of samples and no PK could be determined. It was thus not possible to assess linearity after oral administration. The two oral 20 mg doses (2×10 mg capsule)—TP-02 and TP-03—resulted in different extents of exposure with bioavailability for terlipressin of TP-02 being higher than that of TP-03.

The PK of LVP could be determined for the 2 mg and both 20 mg formulations. The increases between 2 mg and either 20 mg formulation in the arithmetic mean Cmax, AUC(0-t), and AUC(inf) were considerably greater than dose proportional, suggesting nonlinear absorption. The bioavailability for LVP was highest for TP-02, followed by TP-03.

A clear advantage was shown by a formulation containing PVP-12 compared to that containing PVP-K30.

### Example 5: Description of the Octreotide and Terlipressin Capsules Produced

The constituents of a single octreotide capsule and a single terlipressin capsule produced with the improved coating combination are presented in Table 14.

TABLE 14

| | | Octreotide | Terlipressin |
|---|---|---|---|
| | | acetate | acetate |
| | | amount per | amount per |
| | Constituent | capsule (mg) | capsule (mg) |
| API in suspension | API (free-base) in capsule fill mass | 30.0^{b} | 20.00 |
| | as described above | | |
| | Polyvinyl pyrrolidone 12 (PVP-12) | 60.0 | 60.0 |
| | Sodium caprylate (NaC₈) | 91.0 | 91.0 |
| | MgCl2 hexahydrate (anhydrous) | 3.6 | 3.6 |
| | Polysorbate 80 (Tween 80) | 12.0 | 12.0 |
| | Glyceryl monocaprylate (GMC) | 24.0 | 24.0 |
| | Glyceryl tricaprylate (GTC) - | 379.4 | 389.4 |
| | Captex 8000 | | |
| Gelatin capsule | Coni-Snap (CS) hard gelatin | 1 unit = 96 mg | 1 unit = 96 mg |
| | capsules, white, size 0 | | |
| | Gelatin powder 220 Bloom for | 9 mg | 9 mg |
| | banding | | |
| First coating: | 57.0% polyvinyl alcohol-partly | 2.9 | 2.9 |
| OPADRY amb II | hydrolysed (USP, FCC, PhEur, | | |
| clear = 5.1 mg | JPE) | | |
| | 34.0% talc (USP, FCC, PhEur, JP) | 1.7 | 1.7 |
| | 6.0% glycerol monocaprylate Type | 0.3 | 0.3 |
| | 1 (PhEur) | | |
| | 3.0% sodium lauryl sulfate (NF, | 0.2 | 0.2 |
| | PhEur,JP, ChP) | | |
| Second coating: | 97.0% methacrylic acid and ethyl | 38.0 | 38.0 |
| Eudragit L 30 D-55 = | acrylate copolymer dispersion 1:1 | | |
| 39.2 mg | (USP); previously termed in the USP | | |
| | methacrylic acid copolymer type C. | | |
| | 0.7% sodium lauryl sulfate | 0.3 | 0.3 |
| | 2.3% Polysorbate 80 Ph. Eur. / NF | 0.9 | 0.9 |
| Added to Eudragit | Triethyl citrate (TEC) | 8.0 | 8.0 |
| L30 D55 | | | |
| 3^{rd} coating | Talc | 1 | 1 |

^{b}Octreotide acetate or Terlipressin Acetate is used to manufacture the drug product. The amount per capsule corresponds to 30 mg octreotide free peptide or 20 mg Terlipressin free peptide.

Note: Alternatively, capsules may be produced as described above but without the first coating(i.e. without the OPADRY amb II). This results in capsules coated with Eudragit 30 D-55 (plus TEC) only and then talc is applied on top of that coating.

### Embodiments

1. An oral dosage form comprising a capsule containing a therapeutic agent wherein the capsule comprises a first coating comprising polyvinyl alcohol, and further comprises a second coating on top of the first coating comprising methacrylic acid and ethyl acrylate copolymer dispersion (generally termed EudragitL100)
2. The oral dosage form of embodiment 1 where the dispersion is in the range 1.4:1 to 1:1.4 methacrylic acid and ethyl acrylate copolymer.
3. The oral dosage form of embodiment 2 where the dispersion is in the range 1.2:1 to 1:1.2 methacrylic acid and ethyl acrylate copolymer
4. The oral dosage form of embodiment 1 where the dispersion is 1:1 methacrylic acid and ethyl acrylate copolymer.
5. The oral dosage form of embodiment 1 where the polyvinyl alcohol is partially hydrolysed.
6. The oral dosage form of embodiment 1 where the polyvinyl alcohol has molecular weight between 20,000-35,000 and preferably between 26,300 and 30,000.
7. The oral dosage form of embodiment 1 where the methacrylic acid and ethyl acrylate copolymer has molecular weight between 30,000 and 40,000 and preferably about 34000.
8. The oral dosage form of embodiment 1 which further comprises a third coating on top of the second coating which comprises talc.
9. The oral dosage form of embodiment 1 wherein the capsule consists of gelatin for example hard gelatin capsule or HPMC.
10. The oral dosage form of embodiment 1 wherein the second coating additionally comprises sodium lauryl sulfate and polysorbate 80.
11. The oral dosage form of embodiment 1 wherein the second coating does not comprise sodium bicarbonate.
12. The oral dosage form of embodiment 1 wherein the second coating does not comprise titanium dioxide.
13. The oral dosage form of embodiment 1 wherein the first coating additionally comprises talc, glycerol monocaprylocaprate type 1 and sodium lauryl sulfate.
14. The oral dosage form of embodiment 13 wherein the second coating comprises sodium lauryl sulfate and polysorbate 80.
15. The oral dosage form of embodiment 1 wherein the therapeutic agent is a polypeptide
16. The oral dosage form of embodiment 14 wherein the therapeutic agent is a polypeptide
17. The oral dosage form of embodiment 15 wherein the polypeptide is terlipressin or an analog thereof or octreotide or an analog thereof.
18. The oral dosage form of embodiment 16 wherein the polypeptide is terlipressin or an analog thereof or octreotide or an analog thereof.
19. The oral dosage form of embodiment 15 wherein the polypeptide is terlipressin or salt thereof.
20. The oral dosage form of embodiment 15 wherein the polypeptide is octreotide or salt thereof.
21. The oral dosage form of embodiment 15 wherein the oral dosage form is a gelatin capsule.
22. The oral dosage form of embodiment 21 wherein the oral dosage form is a hard gelatin capsule.
23. The oral dosage form of embodiment 19 wherein the terlipressin is present at 5-50 mg per capsule, preferably 10, or 20 or 30 mg.
24. The oral dosage form of embodiment 20 wherein the octreotide is present at 5-50 mg per capsule preferably 10, or 20 or 30 mg.
25. Method of treatment of a subject suffering from hypotension or portal hypertension or variceal bleeding or hepatorenal syndrome or ascites (in particular severe cirrhotic ascites) or a combination thereof which comprises administering to the subject a therapeutically effective amount of the oral dosage form of embodiment 19.
26. Method of treatment of a subject suffering from acromegaly or neuroendocrine tumor which comprises administering to the subject a therapeutically effective amount of the oral dosage form of embodiment 20.
27. Method of treatment of a subject suffering from symptoms of neuroendocrine tumor such as diarrhea and/or flushing which comprises administering to the subject a therapeutically effective amount of the oral dosage form of embodiment 20.
28. The oral dosage form of embodiments 1-5 where the first coating comprises 40-80% polyvinyl alcohol, 20-55% talc, 1-20% glycerol monocaprylate and 1-5% sodium lauryl sulfate and where the second coating comprises 80-99.0% methacrylic acid and ethyl acrylate copolymer, 0.1%-2% sodium lauryl sulfate and 0.5-4% polysorbate and additionally triethyl citrate.
29. The oral dosage form of embodiments 1-5 where the first coating comprises 50-60% polyvinyl alcohol, 30-40% talc, 4-10% glycerol monocaprylate and 2-4% sodium lauryl sulfate and where the second coating comprises 90-99.0% methacrylic acid and ethyl acrylate copolymer, 0.3%-1% sodium lauryl sulfate and 1-3% polysorbate and additionally triethyl citrate.
30. The oral dosage form of embodiments 1-5 where the capsule coating contains as first coating 57.0% polyvinyl alcohol, 34.0% talc, 6% glycerol monocaprylate and 3% sodium lauryl sulfate and where the second coating comprises 97.0% methacrylic acid and ethyl acrylate copolymer, 0.7% sodium lauryl sulfate and 2.3% polysorbate and additionally triethyl citrate.
31. The oral dosage form of embodiments 28-30 wherein the triethyl citrate is present at an amount of 5-30% of the second coating.
32. The oral dosage form of embodiments 28-30 wherein the triethyl citrate is present at an amount of 10-20% of the second coating, preferably about 17% most preferably 16.9% of the second coating (8 mg per capsule).
33. The oral dosage form of embodiments 28-32 which comprises additionally a third coating.
34. The oral dosage form of embodiment 33 wherein the third coating is talc.
35. The oral dosage form of embodiment 34 wherein the talc is present at an amount of 0.1-3 mg per capsule, preferably 0.5-2 mg per capsule most preferably 1 mg per capsule.
36. A method of producing an enteric-coated capsule containing a therapeutic agent which comprises applying to the capsule a first coating which comprises polyvinyl alcohol, talc, glycerol monocaprylate and sodium lauryl sulfate, and further applying a second coating on top of the first coating wherein the second coating comprises methacrylic acid and ethyl acrylate copolymer dispersion, sodium lauryl sulfate, polysorbate and triethyl citrate.
37. The method of embodiment 36 which additionally comprises applying a third coating on top of the second coating wherein the third coating is talc.
38. The method of embodiment 36 wherein the therapeutic agent is a polypeptide,
39. The method of embodiment 38 wherein the polypeptide is terlipressin or octreotide.
40. An oral dosage form comprising a capsule or tablet or sachet containing pellets containing a therapeutic agent wherein the pellets comprise a first coating comprising polyvinyl alcohol, and further comprise a second coating on top of the first coating comprising methacrylic acid and ethyl acrylate copolymer dispersion (generally termed Eudragit L100).
41. The oral dosage form of embodiment 40 wherein the therapeutic agent is a polypeptide
42. The oral dosage form of embodiment 41 wherein the polypeptide is octreotide or terlipressin.
43. The oral dosage form of embodiment 40 wherein the pellets comprise a medium chain fatty acid salt and polyvinylpyrrolidone (PVP).
44. An oral dosage form comprising a capsule containing a formulation comprising a therapeutic agent wherein the capsule comprises a first coating comprising hydroxypropyl methylcellulose (HPMC) or hydroxypropyl cellulose (HPC) or shellac and further comprises a second coating on top of the first coating comprising methacrylic acid and ethyl acrylate copolymer dispersion.
45. The oral dosage form of embodiment 44 wherein the therapeutic agent is a polypeptide
46. The oral dosage form of embodiment 45 wherein the polypeptide is octreotide or terlipressin
47. The oral dosage form of embodiment 44 wherein the formulation comprises a medium chain fatty acid salt and polyvinylpyrrolidone (PVP).
48. The oral dosage form of embodiment 44 which comprises additionally a third coating.
49. The oral dosage form of embodiment 48 wherein the third coating is talc.
50. The method of treatment of a subject which comprising administering any of the oral dosage forms of embodiments 1-26, 28-35 or 40-49 to the subject wherein the dosage is administered once, twice or three times per day.
51. The method of treatment of embodiment 50 wherein the administering occurs at least 1 hour before a meal or at least 2 hours after a meal.
52. The method of treatment of embodiment 50 wherein the administering occurs on an empty stomach.
53. An oral dosage form comprising a capsule comprising a suspension which comprises an admixture of a hydrophobic oily medium and a solid form wherein the solid form comprises a therapeutically effective amount of polypeptide and at least one salt of a medium chain fatty acid at an amount of at least 10% by weight and polyvinylpyrrolidone (PVP) at an amount of at least 3% or more by weight, wherein the capsule comprises a coating comprising methacrylic acid and ethyl acrylate copolymer dispersion (generally termed Eudragit Li00)
54. The oral dosage form of embodiment 53 where the dispersion is in the range 1.4:1 to 1:1.4 methacrylic acid and ethyl acrylate copolymer.
55. The oral dosage form of embodiment 53 where the dispersion is in the range 1.2:1 to 1:1.2 methacrylic acid and ethyl acrylate copolymer
56. The oral dosage form of embodiment 53 where the dispersion is 1:1 methacrylic acid and ethyl acrylate copolymer.
57. The oral dosage form of embodiment 53 where the methacrylic acid and ethyl acrylate copolymer has molecular weight between 30,000 and 40,000 and preferably about 34000.
58. The oral dosage form of embodiment 53 which further comprises another coating on top of the coating which comprises talc.
59. The oral dosage form of embodiment 53 wherein the capsule consists of gelatin for example hard gelatin capsule or HPMC.
60. The oral dosage form of embodiment 53 wherein the coating additionally comprises sodium lauryl sulfate and polysorbate 80.
61. The oral dosage form of embodiment 1 wherein the coating does not comprise sodium bicarbonate.
62. The oral dosage form of embodiment 53 wherein the coating does not comprise titanium dioxide.
63. The oral dosage form of embodiment 53 wherein the therapeutic agent is a polypeptide
64. The oral dosage form of embodiment 60 wherein the therapeutic agent is a polypeptide
65. The oral dosage form of embodiment 63 wherein the polypeptide is terlipressin or an analog thereof or octreotide or an analog thereof.
66. The oral dosage form of embodiment 64 wherein the polypeptide is terlipressin or an analog thereof or octreotide or an analog thereof.
67. The oral dosage form of embodiment 66 wherein the polypeptide is terlipressin or salt thereof.
68. The oral dosage form of embodiment 66 wherein the polypeptide is octreotide or salt thereof.
69. The oral dosage form of embodiment 60 wherein the oral dosage form is a gelatin capsule.
70. The oral dosage form of embodiment 69 wherein the oral dosage form is a hard gelatin capsule.
71. The oral dosage form of embodiment 66 wherein the terlipressin is present at 5-50 mg per capsule, preferably 10, or 20 or 30 mg per capsule.
72. The oral dosage form of embodiment 68 wherein the octreotide is present at 5-50 mg per capsule preferably 10, or 20 or 30 mg per capsule.
73. Method of treatment of a subject suffering from hypotension or portal hypertension or variceal bleeding or hepatorenal syndrome or ascites (in particular severe cirrhotic ascites) or a combination thereof which comprises administering to the subject a therapeutically effective amount of the oral dosage form of embodiment 67.
74. Method of treatment of a subject suffering from acromegaly or neuroendocrine tumor which comprises administering to the subject a therapeutically effective amount of the oral dosage form of embodiment 68.
75. Method of treatment of a subject suffering from symptoms of neuroendocrine tumor such as diarrhea and/or flushing which comprises administering to the subject a therapeutically effective amount of the oral dosage form of embodiment 68.
76. The oral dosage form of embodiments 53-72 where the coating comprises 80-99.0% methacrylic acid and ethyl acrylate copolymer, 0.1%-2% sodium lauryl sulfate and 0.5-4% polysorbate and additionally triethyl citrate.
77. The oral dosage form of embodiments 1-5 where the coating comprises 90-99.0% methacrylic acid and ethyl acrylate copolymer, 0.3%-1% sodium lauryl sulfate and 1-3% polysorbate and additionally triethyl citrate.
78. The oral dosage form of embodiments 1-5 where the capsule coating comprises 97.0% methacrylic acid and ethyl acrylate copolymer, 0.7% sodium lauryl sulfate and 2.3% polysorbate and additionally triethyl citrate.
79. The oral dosage form of embodiments 28-30 wherein the triethyl citrate is present at an amount of 5-30% of the coating.
80. The oral dosage form of embodiments 28-30 wherein the triethyl citrate is present at an amount of 10-20% of the coating, preferably about 17% most preferably 16.9% of the second (8 mg per capsule).
81. The oral dosage form of embodiments 28-32 which comprises an additional coating.
82. The oral dosage form of embodiment 33 wherein the additional coating is talc.
83. The oral dosage form of embodiment 34 wherein the talc is present at an amount of 0.1-3 mg per capsule, preferably 0.5-2 mg per capsule most preferably 1 mg per capsule.
84. A method of producing an enteric-coated capsule containing a therapeutic agent which comprises applying to the capsule a first coating which comprises polyvinyl alcohol, talc, glycerol monocaprylate and sodium lauryl sulfate, and further applying a second coating on top of the first coating wherein the second coating comprises methacrylic acid and ethyl acrylate copolymer dispersion, sodium lauryl sulfate, polysorbate and triethyl citrate
85. The method of embodiment 36 which additionally comprises applying an additional coating on top of the coating wherein the additional coating is talc.
86. The method of embodiment 36 wherein the therapeutic agent is a polypeptide.
87. The method of embodiment 86 wherein the polypeptide is octreotide or terlipressin.
88. Method of treatment of a patient suffering from severe diarrhea and/or flushing episodes associated with metastatic carcinoid tumors by administration to the patient any of the above oral dosage forms wherein the oral dosage form contains a therapeutically effective amount of octreotide.
89. The method of treatment of embodiment 88 wherein the administering occurs at least 1 hour before a meal or at least 2 hours after a meal.
90. The method of treatment of embodiment 88 wherein the administering occurs on an empty stomach.
91. The method of treatment of embodiment 88 wherein the administering is 10-80 mg per day.
92. The method of treatment of embodiment 88 wherein the administering is 10, 20, 30, 40, 50, 60, 70 or 80 mg per day.

1.-102. (canceled)

103. An oral dosage form comprising a capsule comprising a therapeutic agent, wherein the capsule is coated with a first coating comprising polyvinyl alcohol, and a second coating on top of the first coating comprising a methacrylic acid and ethyl acrylate copolymer dispersion.

104. The oral dosage form of claim 103, wherein the ratio of methacrylic acid to ethyl acrylate in the copolymer is 1.4:1 to 1:1.4.

105. The oral dosage form of claim 103, wherein the polyvinyl alcohol is partially hydrolysed.

106. The oral dosage form of claim 103, wherein the polyvinyl alcohol has an average molecular weight between 20,000-35,000 Da.

107. The oral dosage form of claim 103, wherein the methacrylic acid and ethyl acrylate copolymer has an average molecular weight between 30,000 and 40,000 Da.

108. The oral dosage form of claim 103, further comprising a third coating on top of the second coating, wherein the third coating comprises talc.

109. The oral dosage form of claim 103, wherein the capsule consists of i) gelatin or ii) HPMC.

110. The oral dosage form of claim 103, wherein the second coating further comprises sodium lauryl sulfate and polysorbate 80.

111. The oral dosage form of claim 103, wherein the second coating does not comprise sodium bicarbonate or titanium dioxide.

112. The oral dosage form of claim 103, wherein the first coating further comprises talc, glycerol monocaprylocaprate type 1 and sodium lauryl sulfate.

113. The oral dosage form of claim 103, wherein the second coating comprises sodium lauryl sulfate and polysorbate 80.

114. The oral dosage form of claim 103, wherein the therapeutic agent is a polypeptide.

115. The oral dosage form of claim 114, wherein the polypeptide is terlipressin or an analog thereof or octreotide or an analog thereof.

116. The oral dosage form of claim 114, wherein the polypeptide is octreotide or salt thereof.

117. The oral dosage form of claim 115, wherein the oral dosage form comprises 5-50 mg of terlipressin.

118. The oral dosage form of claim 115, wherein the oral dosage form comprises 5-50 mg of octreotide.

119. A method of treating a subject suffering from acromegaly or a neuroendocrine tumor, comprising administering to the subject a therapeutically effective amount of the oral dosage form of claim 116.

120. The oral dosage form of claim 103, wherein the first coating comprises 40-80% (wt %) of polyvinyl alcohol, 20-55% (wt %) of talc, 1-20% (wt %) of glycerol monocaprylate and 1-5% (wt %) of sodium lauryl sulfate and the second coating comprises 80-99.0% (wt %) of the methacrylic acid and ethyl acrylate copolymer, 0.1%-2% (wt %) of sodium lauryl sulfate, 0.5-4% (wt %) of polysorbate and triethyl citrate.

121. The oral dosage form of claim 120, wherein the second coating comprises 5-30% (wt %) of triethyl citrate.

122. A method of producing an enteric-coated capsule comprising a therapeutic agent, wherein the method comprises:
applying to the capsule a first coating which comprises polyvinyl alcohol, talc, glycerol monocaprylate and sodium lauryl sulfate, and
applying a second coating on top of the first coating wherein the second coating comprises a methacrylic acid and ethyl acrylate copolymer dispersion, sodium lauryl sulfate, polysorbate and triethyl citrate.

123. An oral dosage form comprising a capsule, wherein the capsule comprises a formulation comprising a therapeutic agent, wherein the capsule is coated with a first coating comprising hydroxypropyl methylcellulose (HPMC), hydroxypropyl cellulose (HPC) or shellac and a second coating on top of the first coating comprising a methacrylic acid and ethyl acrylate copolymer dispersion.

124. The oral dosage form of claim 123, wherein the therapeutic agent is a polypeptide.

125. The oral dosage form of claim 124, wherein the polypeptide is octreotide or terlipressin.

126. The oral dosage form of claim 123, wherein the formulation comprises a medium chain fatty acid salt and polyvinylpyrrolidone (PVP).

127. An oral dosage form comprising a capsule and a suspension, wherein the suspension comprises an admixture of a hydrophobic oily medium and a solid form wherein the solid form comprises a therapeutically effective amount of a polypeptide and at least one salt of a medium chain fatty acid in an amount of at least 10% by weight and polyvinylpyrrolidone (PVP) in an amount of at least 3% by weight, wherein the capsule is coated with a coating comprising methacrylic acid and ethyl acrylate copolymer dispersion.

128. The oral dosage form of claim 127, wherein the ratio of methacrylic acid to ethyl acrylate in the copolymer is 1.4:1 to 1:1.4.

129. The oral dosage form of claim 127, wherein methacrylic acid and ethyl acrylate copolymer has an average molecular weight between 30,000 and 40,000 Da.

130. The oral dosage form of claim 127, wherein the coating further comprises sodium lauryl sulfate and polysorbate 80.

131. The oral dosage form of claim 127, wherein the therapeutic agent is a polypeptide.
